(19) United States
(12) Patent Application Publication
(10) Pub. No.: US 2009/0131366 A1
(43) Pub. Date: May 21, 2009

(54) Use of Amine-Borane Compounds as Anti-Microbial Agents

(76) Inventors: Morris Srebnik, Mevasseret Zion (IL); Khuloud Takrouri, Jerusalem (IL); Jehoshua Katzhendler, Jerusalem (IL); Itzhack Polacheck, Jerusalem (IL)

Correspondence Address: MARTIN D. MOYNIHAN d/b/a PRTSI, INC., P.O. BOX 16446, ARLINGTON, VA, 22215, US

(21) Appl. No.: 11/991,779

(22) PCT Filed: Sep. 13, 2006
(86) PCT No.: PCT/IL2006/001073

§ 371 Date: Jan. 2, 2009

Related U.S. Application Data
(60) Provisional application No. 60/716,082, filed on Sep. 13, 2005.

(51) Int. Cl. A61K 31/69; A61P 31/04; A61P 31/10; A61P 33/02

(52) U.S. Cl. 514/64

(57) ABSTRACT

Use of amine-borane compounds such as amine cyanoboranes and amino carboxy boranes in the treatment of medical conditions associated with pathogenic microorganisms and particularly against drug-resistant microorganisms, in the treatment of fungal and protozoal infections is disclosed. Use of amine-borane compounds for reducing the load of microorganisms in various substrates and products is further disclosed.

### FIELD AND BACKGROUND OF THE INVENTION

The present invention relates to novel methods of treating diseases and infections caused by a pathogenic microorganism and of reducing microorganisms load in various substrates.

In the past few decades, amine-borane compounds have been considered as highly sought synthetic targets. Amine-borane compounds exhibit high similarity to organic compounds mainly due to the atomic radii and the characteristics of the B—N bond, which resemble those of carbon-carbon bonds. Thus, for example, a H₂B—NH₂ bond resembles a H₂C═CH₂ double bond, while a H₃B←NH₃ bond resembles a H₃C—CH₃ single bond.

Amine-borane compounds such as, for example, α-aminoboronic acids, amine-carboxyboranes, amine-cyanoboranes, and related compounds are therefore isoelectronic and isostructural analogs of many biologically active compounds such as amino acids, neurotransmitters, nucleosides, and nucleic acids and hence can mimic the biological activity of such compounds in the body. Doing so, these boron compounds may act as inhibitors, antagonists and otherwise effectors of many biological systems and hence have been widely recognized as highly potential therapeutic agents.

For example, α-aminoalkyl boronic acids are analogs of α-amino acids, which may act as inhibitors of enzymes involved in amino acid and peptide metabolism. α-aminoalkyl boronic acids, in which the carboxyl group of the corresponding amino acids is replaced by a boronic acid function, constitute a unique class of amino acid mimics from which a number of potent enzyme inhibitors were synthesized. The inhibitory activity of such compounds mainly stems from the fact that the tetrahedral adduct of electrophilic boronic acid is a good mimic of the putative tetrahedral transition state or intermediate encountered in the enzymatic hydrolysis or formation of peptides. Since the peptide hydrolysis and formation invariably involves the tetrahedral high energy species in the course of the reaction, these amino acid mimics serve as a general key element for inhibitors of a broad spectrum of proteases and peptide ligases.

Additional amine-boranes and compounds having a bis-borane, and mainly cyano and carboxy borane derivatives of these families are disclosed, for example, in U.S. Pat. Nos. 4,301,129, 4,312,989, 4,368,194, 4,550,186, 4,647,555, 4,658,051, 4,740,504, 4,774,354, 4,855,493, 4,977,268, 5,280,119 and 5,312,816 and in Hall, I. H. et al., J. Pharm. Sci. 1980, 69(9), 1025.; Sood, C. K. et al., J. Pharm. Sci. 1991, 80(12), 1133; Dembitsky, V. M., et al., Tetrahedron 2003, 59, 579; E. Shalom et al., Organometallics. 2004, 23, 4396-4399; Berdy, J., Handbook of Antibiotic Compounds. Part IV, CRC, 1980; Fink, K. et al., Science, 1948, 108, 358-9; Hunt, S., Methods Enzymol. 1984, 107, 413-438; Jimenez, E. C. et al., Biochemistry 1997, 36, 984-989; Takrouri et al., Organometallics, 2004, 23(11), 2817-2820; and Gyoeri, B. et al., Inorganic Chemistry, 1998, 37(20), 5131-5141.

The first amine-cyanoboranes and amine-carboxyboranes which stood-out as pharmacologically promising compounds in the early 1980's [1] were adducts of tertiary low-alkyl ammonium salts, typically trimethylammonium chloride, and sodium cyanoborane. Further derivatization of this basic form afforded amines-boranes with aromatic, heterocyclic and silyl substituents on the amine, substitutions of the boron by low-alkyls and bromine, and esters of amine-carboxyboranes [2].

Model studies had shown that these compounds have potent anticancer activity [3, 4-8], antihyperlipodemic activity [910], anti-obesity activity [9], antiosteoporotic activity [9, 11], anti-inflammatory activity [9, 1, 12], hypolipidemic activity [5, 13], anti-neoplastic activity [14-15] and other promising biological activities [16], yet their exact mechanism of action is still not fully understood.

While amine-borane compounds such as those described hereinabove were found beneficially active in various therapeutic applications, their use in the treatment of diseases and infections associated with pathogenic microorganisms have not been taught hitherto. Such diseases and infections have recently become a major worldwide health threat.

Cancerous cells and fungal cells share many traits of primitive eukaryotic cells having similar metabolism which is different from the host cells (e.g., higher growth rate, higher multiplication). The part of the host immune system which suppresses cancer cells is also responsible for the suppression of fungal cells. Under specific circumstances both cancer and fungal cells are not responsive to the innate neural/hormonal control mechanism of the host, resulting in infinite unregulated growth.

The incidence of fungal infections and mycoses has increased significantly in the past two decades mainly due to the growing number of immunocompromised patients such as cancer patients, patients who have undergone organ transplantation, and patients with AIDS, as well as due to the frequent use of cytotoxic and/or antibacterial drugs, which alter the normal bacterial flora.

Fungi include moulds, yeasts and higher fungi. All fungi are eukaryotic and have sterols but not peptidoglycan in their cell membrane. They are chemoheterotrophs (requiring organic nutrition) and most are aerobic. Many fungi are also saprophytes (living off dead organic matter) in soil and water and acquire their food by absorption. Characteristically they also produce sexual and asexual spores. There are over 100,000 species recognized, with 100 infectious members for humans.

Human fungal infections are uncommon in generally healthy persons, being confined to conditions such as candidiasis (thrush) and dermatophyte skin infections such as athlete's foot. Nevertheless, yeast and other fungi infections are one of the human ailments which still present a formidable challenge to modern medicine. In an immunocompromised host, a variety of normally mild or nonpathogenic fungi can cause potentially fatal infections. Furthermore, the relative ease with which human can now travel around the world provides the means for unusual fungal infections to be imported from place to place. Therefore, wild and resistant strains of fungi are considered to be one of the most threatening and frequent cause of death mainly in hospitalized persons and immunocompromised patients.

Resistance of microorganism to antimicrobial agents is the ability of a microorganism to withstand the antimicrobial effects of any given agents (antibiotics). The antimicrobial action of any given agent is putting an environmental pressure on the target (and also non-targeted) microorganisms. The microorganisms which have a mutation that will allow it to survive will live on to reproduce. These newly evolved strain(s) will then pass this trait to their offspring, which will constitute a fully resistant generation. Resistance can develop naturally via natural selection through random mutation and programmed evolution governed by low-fidelity polymerases which can cause a higher rate of random mutations in the microorganism genetic code. Once such a gene is generated, the microorganism may also transfer the genetic information in a horizontal fashion, namely between individuals, via plasmid exchange, hence resistance is a consequence of evolution via natural selection or programmed evolution.

It has been demonstrated that habitude of antimicrobial agents usage greatly affect the number of resistant organisms which develop. For example, overuse of broad-spectrum antibiotics of low specificity, such as second- and third-generation cephalosporins and fungicides such as fluconazole, greatly accelerated the development of methicillin or fluconazole resistance, and increase selection of pre-existing resistant strains that have never been exposed to the selective pressure of methicillin or fluconazole per se. Other factors contributing to the ever growing emergence of resistance to antimicrobials include incorrect diagnosis, unnecessary prescriptions, improper use of antimicrobials by patients, increasing use of prophylaxis and suppression therapy and the use of antimicrobials in livestock food as additives for growth promotion.

In the 1990s, there was a significant increase in the prevalence of drug-resistant fungal infections due to non-Candida species in patients hospitalized for mucosal or systemic diseases. The widespread application of fluconazole or related azole antifungal agents is postulated to promote selection of resistant subpopulations by shifting colonization to more naturally resistant species, such as Candida krusei or Candida glabrata.

For example, Candida vaginitis is a common problem attributable to overgrowth of Candida species. It is estimated that 75% of all women will experience an episode in their lifetime. By the age of 25 years, nearly one-half of all women will have had at least one episode of Candida vaginitis. Candida albicans accounts for 80% to 95% of all episodes of Candida vaginitis worldwide. Like other topical Candida infections, Candida vaginitis is treated effectively with azole-based antifungal drugs. However, such therapy can be complicated by the emergence of drug-resistant yeasts. Prolonged exposure to fluconazole can shift the predominant vaginal yeast flora from C. albicans to more intrinsically azole-resistant species, as has been described for immunosuppressed women.

Only a small number of anti-fungal agents is presently available, whereby all are associated with one or more drawbacks. Until recently, amphotericin B was the standard therapy for many fungal infections, but a high frequency of renal toxicity has limited its use. The use of azoles and triazoles, such as fluconazole, has increased the ability to treat many fungal infections. However, mortality due to these infections, even with antifungal therapy, is still unacceptably high.

There are several causes for the laggardness of the anti-fungal medicine, broadly stemming from enhanced emergence of drag-resistant pathogenic strains, lack of drug specificity and limitations associated with the spectrum of activity of the drugs and other general pharmacokinetic weaknesses

Besides the increasing number of fungal infections, stemming from the abovementioned causes such as use of immunosuppressing treatments, autoimmune diseases and the unhindered use of antimicrobial agents, there is also an emergence of resistance to antifungal agents amongst strains which were affected in the past, oftentimes due to antimicrobial treatment which was not directed initially against fungi. Some disadvantages are linked to the spectrum of activity of these agents, i.e., lethal to beneficial microorganisms and/or ineffective towards pathogenic fungi. Some drawbacks stem from a poor or inefficient pharmacokinetic profile, which may be associated with high toxicity index. Other problems with currently available anti-fungal agents are associated with tissue distribution, and especially CNS penetration. In some cases lipid formulations may solve the tissue distribution problems, but these treatments are prohibitively expensive, as in the case of amphotericin B.

Another class of pathogenic microorganisms which still baffles modern medicine includes parasites and protozoa such as, for example, those which cause malaria and leishmaniasis.

These parasites and protozoa afflict more than 500 million people per year, mostly in the tropics and subtropics. The resulting diseases cause disability, disfigurement, and in some cases death. Vaccines against these single cell organisms seem inefficient due to antigenic variation. Additionally, parasites and protozoa promote drug resistance via multiple pathways. The drugs currently available to destroy these parasites are toxic to humans themselves.

Malaria, also called jungle fever, paludism and swamp fever, is an infectious disease characterized by cycles of chills, fever, and sweating, caused by the parasitic infection of red blood cells by the protozoan parasite, Plasmodium (one of the Apicomplexa family), which is transmitted by the bite of an infected vector for human malarial parasite, a female Anopheles mosquito. Of the four types of malaria, the most life-threatening type is falciparum malaria. The other three types of malaria, vivax, malariae, and ovale, are generally less serious and are not life-threatening.

Malaria is probably the deadliest infectious disease yet to be beaten, causing about half a billion infections and between one and two millions deaths annually, mainly amongst children in the tropics and sub-Saharan Africa. The Plasmodium falciparum variety of the parasite accounts for 80% of cases and 90% of deaths. The stickiness of the red blood cells is particularly pronounced in P. falciparum malaria and this is the main factor giving rise to hemorrhagic complications of malaria.

To date there is no absolute cure for malaria. Malaria eradication has been hampered by the development of Plasmodia resistant to currently available antimalarial drugs, especially P. falciparum, which is the most abundant and dangerous causative species. Since no antimalarial vaccine is available to date, the control of this deadly disease presently relies on pharmacological treatment. If diagnosed early, malaria can be alleviated, but prevention is still more effective than treatment. Since the 17^{th} century quinine has been the prophylactic of choice for malaria. The development of quinacrine, chloroquine, and primaquine in the 20^{th} century reduced the reliance on quinine. These anti-malarial medications can be taken preventively, which is commonly recommended for travelers to affected regions.

Unfortunately as early as the 1960s several strains of the malarial parasite developed resistance to chloroquine. This development of resistance, plus the growing immunity of mosquitoes to insecticides, has caused malaria to become one the of world's leading re-emerging infectious diseases. Mefloquine may be used in areas where the disease has become highly resistant to chloroquine, but some strains are now resistant also to this and other drugs. Artemisinin (derived from sweet wormwood) in combination with other drugs is now in many cases the preferred treatment for resistant strains. Malarone (atovaquone and proguanil) is also used for resistant strains. Vaccines against malaria are still experimental.

Leishmaniasis is a disease which is endemic in large regions of the world including the Middle East and Mediterranean areas. Recently it spreads as an opportunistic disease in HIV patients. There are three broad types of leishmaniasis: coetaneous (CL), mucocutaneous (MCL) and visceral (VL) leishmaniasis. CL is a skin disease which is clinically dangerous following immunosuppression while the other types are lethal if untreated. The World Health Organization estimates the number of Leishmania infections, as more than 400,000 new cases annually. MCL and VL, cause the death of more than 75,000 people annually. The current treatment of leishmaniasis is based on known anti-fungal agents such as amphotericin B, and therapies comprising the use of antimony, antimoniate de meglumine (Glucantime) and sodium stibogluconate (Pentostam). Unfortunately these treatments are inefficient or otherwise inadequate due to limited availability of effective parenteral drug formulations and the appearance of new strains resistant to the marketed drugs.

Other pathogenic protozoa which still plague humanity include Cryptosporidium parvum, Cyclospora cayetanensis and Giardia lamblia. Cryptosporidium parvum which is a protozoan parasite associated with municipal water supplies which causes diarrhea. In patients with a normal immune system, the disease manifests itself with watery diarrhea, cramps, nausea and anorexia, lasting ten to fifteen days. In immunocompromised patients, such as those receiving immunosuppressant drugs or those infected with HIV-1, symptoms are more severe. The disease is prolonged, and diarrhea can persist for months, even years. Cyclospora cayetanensis infections result in a disease with non-specific symptoms. In general, there is usually one day of malaise, low fever and diarrhea. There may be fatigue, vomiting and weight loss. The disease usually is self-limiting in three to four days, but diarrhea relapses may occur for up to four weeks. Human giardiasis (“beaver fever”) usually results from drinking water contaminated with the protozoan Giardia lamblia (also known as G. intestinalis). Infections are frequently seen in day care centers and among campers. In acute cases, symptoms may include nausea, upper intestinal pain and explosive diarrhea. Fever and chills may be present, and in fact, symptoms may mimic a peptic ulcer or gall bladder disease.

Hence, while diseases and infections caused by the above-cited microorganisms have become a major health threat, the presently available treatments thereof are becoming less and less efficient.

There is thus a widely recognized need for, and it would be highly advantageous to have novel agents for treating the above-mentioned and other diseases associated with pathogenic microorganisms devoid of the above limitations.

### SUMMARY OF THE INVENTION

The present inventors have now surprisingly uncovered that amine-borane compounds can act as antimicrobial agents and can thus be used in the treatment of various medical conditions, as well as in other, non-medical applications, which are associated with microorganisms.

Thus, according to one aspect of the present invention there is provided a method of treating a medical condition associated with a pathogenic microorganism, the method comprising administering to a subject in need thereof a therapeutically effective amount of an amine-borane compound.

According to another aspect of the present invention there is provided a use of an amine-borane compound in the treatment of a medical condition associated with a pathogenic microorganism.

According to still another aspect of the present invention there is provided a use of an amine-borane compound for the preparation of a medicament for the treatment of a medical condition associated with a pathogenic microorganism.

According to yet another aspect of the present invention there is provided a pharmaceutical composition identified for use in the treatment of a medical condition associated with a pathogenic microorganism, which includes as an active ingredient, an amine-borane compound and a pharmaceutically acceptable carrier.

According to still further features of the preferred embodiments of the invention described below, the composition is packaged in a packaging material and identified in print, in or on said packaging material, for use in the treatment of said medical condition.

According to still further features in the described preferred embodiments the pathogenic microorganism is selected from the group consisting of a prokaryotic organism, an eubacterium, an archaebacterium, a eukaryotic organism, a yeast, a fungus, an alga, a protozon and a parasite.

According to still further features in the described preferred embodiments the microorganism is a drug-resistant pathogenic microorganism, preferably a drug-resistant fungus.

According to still further features in the described preferred embodiments the amine-borane compound is administered either per se or as a part of a pharmaceutical composition, as described herein.

According to still further features in the described preferred embodiments the medical condition is selected from the group consisting of a bacterial infection, a fungal infection, a protozoan infection, malaria and leishmaniasis.

According to still another aspect of the present invention there is provided a method of treating a medical condition associated with a pathogenic, drug-resistant, microorganism, the method is effected by administering to a subject in need thereof a therapeutically effective amount of an amine-borane compound presented herein.

According to still another aspect of the present invention there is provided a use of an amine-borane compound in the treatment of a medical condition associated with a pathogenic, drug-resistant microorganism.

According to still another aspect of the present invention there is provided a use of an amine-borane compound for the preparation of a medicament for the treatment of a medical condition associated with a pathogenic, drug-resistant microorganism.

According to still another aspect of the present invention there is provided a pharmaceutical composition identified for use in the treatment of a medical condition associated with a pathogenic, drug-resistant microorganism comprising, as an active ingredient, an amine-borane compound according to the present invention and a pharmaceutically acceptable carrier.

According to further features in the described preferred embodiments the pathogenic drug-resistant microorganism is selected from the group consisting of a prokaryotic organism, an eubacterium, an archaebacterium, a eukaryotic organism, a yeast, a fungus, an alga, a protozoon and a parasite., and is preferably a drug-resistant fungus.

According to still further features in the described preferred embodiments the pathogenic, drug-resistant microorganism is resistant to at least one conventional antimicrobial agent.

According to still further features in the described preferred embodiments the conventional antimicrobial agent is selected from the group consisting of a polyene-based antifungal agent, amphotericin, amphotericin B, nystatin, pimaricin, amphotericin B liposomal formulations (AmBisome, Abelcet, Amphocil), an azole-based antifungal agent, fluconazole, itraconazole, ketoconazolean voriconazole, posaconazole clotrimazole, miconazole allylamine- and a morpholine-based antifungal agent, allylamines (naftifine, terbinafine), an antimetabolite-based antifungal agent, 5-fluorocytosine, fungal cell wall inhibitor, caspofingin, micafingin, anidulafingin.

According to still further features in the described preferred embodiments the amine-borane compound is administered either per se or as a part of a pharmaceutical composition, said pharmaceutical composition further comprises a pharmaceutically acceptable carrier.

According to still further features in the described preferred embodiments the medical condition is selected from the group consisting of a bacterial infection, a fungal infection, a protozoan infection, malaria and leishmaniasis.

According to an additional aspect of the present invention there is provided a method of reducing the load of a microorganism in a substrate, the method comprising applying to the substrate an antimicrobial effective amount of an amine-borane compound.

According to further features in preferred embodiments of the invention described below, the substrate is selected from the group consisting of a construction, a storage container, a soil, an agricultural crop, a horticultural crop, an agricultural product, a food product, a cosmetic product, a paint, a lumber and a building material.

According to still an additional aspect of the present invention there is provided an article-of-manufacturing comprising a product and an antimicrobial effective amount of an amine-borane compound.

According to further features in preferred embodiments of the invention described below, the product is selected from the group consisting of a food product, an agricultural product, a cosmetic product, a paint, a building material and a lumber.

According to still further features in the described preferred embodiments each of the amine-borane compounds described herein has the general Formula I or II:

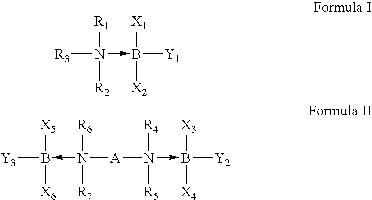

or a pharmaceutically acceptable salt thereof,

wherein:

Y₁, Y₂ and Y₃ are each independently selected from the group consisting of a cyano group (—C≡N), a —C(═O)Ra group, amine and alkyl, whereas Ra is selected from the group consisting of hydrogen, halo, hydroxy, alkoxy, thiohydroxy, thioalkoxy, aryloxy, thioaryloxy, thiol and amine;

X₁-X₆ are each independently selected from the group consisting of hydrogen, alkyl, halo, cycloalkyl, and aryl;

R₁-R₇ are each independently selected from the group consisting of hydrogen, alkyl, alkenyl, alkynyl, cycloalkyl and aryl or, alternatively, two of R₁-R₃, R₄ and R₅ and/or R₆ and R₇ form a carbocyclic ring; and

A is a substituted or non-substituted, saturated or non-saturated hydrocarbon having from 1 to 20 carbon atoms.

According to still further features in the described preferred embodiments the amine-borane compound has the general Formula I.

According to still further features in the described preferred embodiments Y₁ is selected from the group consisting of a cyano group (—C≡N) and a —C(═O)Ra group.

According to still further features in the described preferred embodiments Ra is selected from the group consisting of hydrogen and alkoxy.

According to still further features in the described preferred embodiments the alkoxy is selected from the group consisting of methoxy and ethoxy.

According to still further features in the described preferred embodiments X₁ and X₂ are each independently selected from the group consisting of hydrogen and halo.

According to still further features in the described preferred embodiments at least one of X₁ and X₂ is halo.

According to still further features in the described preferred embodiments the halo is selected from the group consisting of fluoro and bromo.

According to still further features in the described preferred embodiments each of R₁-R₃ is alkyl.

According to still further features in the described preferred embodiments the alkyl is selected from the group consisting of methyl, ethyl and n-butyl.

According to still further features in the described preferred embodiments at least one of R₁-R₃ is a C₅-C₂₀ alkyl.

According to still further features in the described preferred embodiments the amine-borane compound is selected from the group consisting of 1-dimethylaminomethyl-cyclopent-2-enol cyanoborane, (2-hydroxy-2-phenyl-ethyl)-dimethyl-amine cyanoborane, ethyl-dimethyl-amine cyanoborane, but-3-enyl-dimethyl-amine cyanoborane, trimethyl-amine cyanodibromoborane, trimethyl-amine cyanoborane, butyl-dimethyl-amine cyanoborane, pentyl-dimethyl-amine cyanoborane, dimethyl-undecyl-amine cyanoborane, dimethyl-undecyl-amine cyanobromoborane, dimethyl-undecyl-amine cyanodibromoborane, dimethyl-trimethylsilanylmethyl-amine cyanoborane, dodecyl-dimethyl-amine cyanoborane, 1-dimethylamino-2-methyl-octan-2-ol cyanoborane, dimethyl-nonyl-amine cyanoborane, dimethyl-tridecyl-amine cyanoborane, dimethyl-pentadecyl-amine cyanoborane, heptadecyl-dimethyl-amine cyanoborane, 1-dimethylamino-dodecan-2-ol cyanoborane, hex-5-enyl-dimethyl-amine cyanoborane and 1-dimethylamino-undecan-2-ol cyanoborane.

According to still further features in the described preferred embodiments the amine-borane compound is selected from the group consisting of dimethyl-undecyl-amine cyanofluorobromoborane, trimethyl-amine cyanofluoroborane, ethyl-dimethyl-amine cyanofluoroborane, butyl-dimethyl-amine cyanofluoroborane, trimethyl-amine carboxyfluoroborane methyl ester, trimethyl-amine carboxyfluoroborane ethyl ester, ethyl-dimethyl-amine carboxyfluoroborane methyl ester, butyl-dimethyl-amine carboxyfluoroborane methyl ester, trimethyl-amine cyanodifluoroborane, trimethyl-amine carboxydifluoroborane methyl ester, trimethyl-amine carboxydifluoroborane ethyl ester, trimethyl-amine cyanofluorobromoborane, trimethyl-amine carboxyfluorobromoborane ethyl ester, triethyl-amine carboxydifluoroborane, dimethyl-undecyl-amine cyanofluoroborane and (2-fluoro-nonyl)-dimethyl-amine cyanoborane.

According to still further features in the described preferred embodiments the amine-borane compound has the general Formula II.

According to still further features in the described preferred embodiments each of X₃, X₄, X₅ and X₆ is independently selected from the group consisting of hydrogen and halo.

According to still further features in the described preferred embodiments at least one of X₃-X₆ is halo.

According to still further features in the described preferred embodiments the halo is selected from the group consisting of fluoro and bromo.

According to still further features in the described preferred embodiments each of R₄-R₇ is alkyl, preferably methyl.

According to still further features in the described preferred embodiments A is a saturated, non-substituted hydrocarbon.

According to still further features in the described preferred embodiments the hydrocarbon has from 1 to 20 carbon atoms.

According to still further features in the described preferred embodiments the amine-borane compound is selected from the group consisting of N,N,N′,N′-tetramethyl-decane-1,10-diamine cyanoborane, N,N,N′,N′-tetramethyl-decane-1,10-diamine bis-cyanobromoborane, N,N,N′,N′-tetramethyl-decane-1,10-diamine bis-cyanodibromoborane, N,N,N′,N′-tetramethyl-decane-1,10-diamine bis-carboxyborane, N,N,N′,N′-tetramethyl-dodecane-1,12-diamine bis-cyanoborane and N,N,N′,N′-tetramethyl-tetradecane-1,14-diamine bis-cyanoborane.

The present invention successfully addresses the shortcomings of the presently known configurations by providing novel anti-microbial agents that are superior to the presently known agents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used, herein interchangeably and are meant to include the first and second indicated numbers and all the fractional and integral numerals therebetween.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents a plot showing the anti lishmenial effect of Compound K-I as determined by a dose-response assay against the Leishmania donovani strain; and

FIG. 2 presents a plot showing the antimalarial effect of Compound K-I as determined by a dose-response assay against the Plasmodium falciparum strain.

### DESCRIPTION OF THE PREFERRED EMBODIMENTS

The present invention is of novel methods of treating various medical conditions associated with pathogenic microorganisms, which utilize amino-borane compounds such as amine cyanoboranes and amine carboxyboranes.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As discussed hereinabove, diseases and infections caused by microorganisms pause a major health risk. The development of resistance to the presently known medications and the growing number of immunocompromised hosts have prompted a massive search for novel antimicrobial agents.

While conceiving the present invention, the present inventors have envisioned that amine-borane compounds could serve as efficient antimicrobial agents.

As used herein, the phrase “amine-borane compounds”, which is also refereed to herein interchangeably as “aminoboranes”, describes any compound that includes at least one boron atom that is substituted by one or more amine group(s), as is defined hereinunder.

As mentioned above, amine-borane compounds have drawn a considerable interest as pharmaceutical compounds, by being isoelectronic and isostructural analogs of many biologically active compounds such as amino acids, neurotransmitters, nucleosides, and nucleic acids, and hence capable of mimicking the biological activity of such biologically active compounds in the body.

While reducing the present invention to practice, as is demonstrated in the Examples section that follows, it was indeed shown that various, structurally diverse, aminoborane compounds exhibit exceptional and selective antimicrobial activity, mostly in a dose-dependent manner. It was further shown that these compounds are non-toxic at the active concentration range thereof.

Thus, according to one aspect of the present invention, there is provided a method of treating a medical condition associated with a pathogenic microorganism, and preferably pathogenic microorganism which are resistant to antimicrobial agents. The method, according to this aspect of the present invention, is effected by administering to a subject in need thereof a therapeutically effective amount of an amine-borane compound.

According to further aspects of the present invention, uses of amino borane compounds for treating, and for preparing a medicament for treating, a medical condition associated with a pathogenic microorganism, and preferably pathogenic microorganism which are resistant to antimicrobial agents, are provided.

As used herein, the terms “treating” and “treatment” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the phrase “therapeutically effective amount” describes an amount of the compound being administered which will relieve to some extent one or more of the symptoms of the condition being treated.

Herein throughout, the phrase “pathogenic microorganism” is used to describe any microorganism which can cause a disease or infection in a higher organism, such as any animals grown for commercial or recreational purposes, fish, poultry, insects (e.g., bees) and mammals. In particular, the pathogenic microorganism maybe those which cause diseases and adverse effects in humans.

The pathogenic microorganism may belong to any family of organisms such as, but not limited to, prokaryotic organisms, eubacterium, archaebacterium, eukaryotic organisms, yeast, fingi, algae, protozoa, and other parasites.

As is demonstrated in the Examples section that follows, amine-borane compounds were found highly efficient agents against a wide spectrum of microorganisms.

Non-limiting examples of pathogenic microorganism that are treatable by amine borane compounds include Plasmodium falciparum and related malaria-causing protozoan parasites, Acanthamoeba and other free-living amoebae, Aeromonas hydrophila, Anisakis and related worms, Ascaris lumbricoides, Bacillus cereus, Campylobacter jejuni, Clostridium botulinum, Clostridium perfringens, Cryptosporidium parvum, Cyclospora cayetanensis, Diphyllobothrium, Entamoeba histolytica, Eustrongylides, Giardia lamblia, Listeria monocytogenes, Nanophyetus, Plesiomonas shigelloides, Salmonella, Shigella, Staphylococcus aureus, Streptococcus, Trichuris trichiura, Vibrio cholerae, Vibrio parahaemolyticus, Vibrio vulnificus and other vibrios, Yersinia enterocolitica and Yersinia pseudotuberculosis.

Representative examples of pathogenic fungi, against which amine-borane compounds can be efficiently used according to the present embodiments include, without limitation, fungi of the genus Absidia: Absidia corymbifera; genus Ajellomyces: Ajellomyces capsulatus, Ajellomyces dermatitidis; genus Arthroderma: Arthroderma benhamiae, Arthroderma fulvum, Arthroderma gypseum, Arthroderma incurvatum, Arthroderma otae, Arthroderma vanbreuseghemii; genus Aspergillus: Aspergillus flavus, Aspergillus fumigatus, Aspergillus niger; genus Blastomyces: Blastomyces dermatitidis; genus Candida: Candida albicans, Candida glabrata, Candida guilliermondii, Candida krusei, Candida parapsilosis, Candida tropicalis, Candida pelliculosa; genus Cladophialophora: Cladophialophora carrionii; genus Coccidioides: Coccidioides immitis; genus Cryptococcus: Cryptococcus neoformans; genus Cunninghamella: Cunninghamella sp.; genus Epidermophyton: Epidermophyton floccosum; genus Exophiala: Exophiala dermatitidis; genus Filobasidiella: Filobasidiella neoformans; genus Fonsecaea: Fonsecaea pedrosoi; genus Fusarium: Fusarium solani; genus Geotrichum: Geotrichum candidum; genus Histoplasma: Histoplasma capsulatum; genus Hortaea: Hortaea werneckii; genus Issatschenkia: Issatschenkia orientalis; genus Madurella: Madurella grisae; genus Malassezia: Malassezia furfur, Malassezia globosa, Malassezia obtusa, Malassezia pachydermatis, Malassezia restricta, Malassezia slooffiae, Malassezia sympodialis; genus Microsporum: Microsporum canis, Microsporum fulvum, Microsporum gypseum; genus Mucor: Mucor circinelloides; genus Nectria: Nectria haematococca; genus Paecilomyces: Paecilomyces variotii; genus Paracoccidioides: Paracoccidioides brasiliensis; genus Penicillium: Penicillium marneffei; genus Pichia, Pichia anomala, Pichia guilliermondii; genus Pneumocystis: Pneumocystis carinii; genus Pseudallescheria: Pseudallescheria boydii; genus Rhizopus: Rhizopus oryzae; genus Rhodotorula: Rhodotorula rubra; genus Scedosporium: Scedosporium apiospermum; genus Schizophyllum: Schizophyllum commune; genus Sporothrix: Sporothrix schenckii; genus Trichophyton: Trichophyton mentagrophytes, Trichophyton rubrum, Trichophyton verrucosum, Trichophyton violaceum; and of the genus Trichosporon: Trichosporon asahii, Trichosporon cutaneum, Trichosporon inkin, Trichosporon mucoides.

As discussed hereinabove, resistance of microorganism to antimicrobial agents is the ability of a microorganism to withstand the antimicrobial effects of any given agents (antibiotics). The antimicrobial action of any given agent is putting an environmental pressure on the target (and also non-targeted) microorganisms. The microorganisms which have a mutation that will allow it to survive will live on to reproduce. These newly evolved strain(s) will then pass this trait to their offspring, which will constitute a fully resistant generation.

As demonstrated in the Example section that follows, the amine borane compounds presented herein were shown to be particularly effective against fugal strains which developed, or were found intrinsically resistant to conventional antifungal agents such as amphotericin B and fluconazole. Since these strains are also associated with severe medical conditions, the use of these compounds may be one of the few means to combat these pathogens and ameliorate or cure the medical conditions associated therewith.

Hence, there are provided: a method of treating a medical condition associated with a pathogenic, drug-resistant microorganism; use of amino borane compounds for treating a medical condition associated with a pathogenic, drug-resistant microorganism; use of amino borane compounds for preparing a medicament for treating a medical condition associated with a pathogenic, drug-resistant microorganism; and a pharmaceutical composition identified for use in the treatment of a medical condition associated with a pathogenic, drug-resistant microorganism.

The pathogenic, drug resistant microorganism can be any of the microorganisms selected from the group consisting of prokaryotic organisms, eubacteria, archaebacteria, eukaryotic organisms, yeast, fungi, algae, protozoa and other parasites.

The drug-resistant microorganism can be resistant to one or more conventionally used drug or other antimicrobial agent. The phrase “conventional antimicrobial agent” as used herein refers to typically used drugs and antimicrobial agents which are commonly used prior to the disclosure of the present invention, or drugs and antimicrobial agents which are not based on amine-borane as presented herein.

Non-limiting examples of conventional antimicrobial agent include polyene-based antifungal agents such as amphotericin, amphotericin B, nystatin and pimaricin, amphotericin B liposomal formulations (AmBisome, Abelcet, Amphocil), azole-based antifungal agents such as fluconazole, itraconazole and ketoconazole, allylamine- or morpholine-based antifungal agents such as allylamines (naftifine, terbinafine), and antimetabolite-based antifungal agents such as 5-fluorocytosine, and fungal cell wall inhibitor such as echinocandins like caspofungin, micafungin and anidulafingin.

As is further demonstrated in the Examples section that follows, amine-borane compounds were found effective against two life threatening protozoa parasites, Leishmania spp and Plasmodium falciparum. Other representative examples of pathogenic parasites and protozoa, against which amine-boranes can be used according to the present embodiments include, without limitation, various types of amoeba, Trypanosoma cruzi (causing Chagas' disease), Trypanosoma bucei (causing “sleeping sickness”), Plasmodium vivax (causing malaria), Cryptosporidium parvum (causing cryptosporidiosis), Cyclospora cayetanensis, Giardia lamblia (causing giardiasis) and many others.

As used herein the term “associated” in the context of the present invention means that at least one adverse manifestation of the medical condition is caused by a pathogenic microorganism. The phrase “medical condition associated with a pathogenic microorganism” therefore encompasses medical conditions of which the microorganism may be the primary cause of the medical condition or a secondary effect of the main medical condition(s).

Medical conditions associated with a pathogenic microorganism include infections, infestation, contaminations and transmissions by or of pathogenic microorganisms such as those described herein. In general, a disease causing infection is the invasion into the tissues of a plant or an animal by pathogenic microorganisms. The invasion of body tissues by parasitic worms and other higher pathogenic organisms such as lice is oftentimes referred to as infestation.

Invading organisms such as bacteria typically produce toxins that damage host tissues and interfere with normal metabolism; some toxins are actually enzymes that break down host tissues. Other bacterial substances may inflict their damage by destroying the host's phagocytes, rendering the body more susceptible to infections by other pathogenic microorganisms. Substances produced by many invading organisms cause allergic sensitivity in the host. Infections may be spread via respiratory droplets, direct contact, contaminated food, or vectors, such as insects. They can also be transmitted sexually and from mother to fetus.

Examples of medical conditions and diseases caused by bacterial infections, which are treatable by amine-borane compounds according to the present embodiments, include, without limitation, actinomycosis, anthrax, aspergillosis, bacteremia, bacterial skin diseases, bartonella infections, botulism, brucellosis, burkholderia infections, campylobacter infections, candidiasis, cat-scratch disease, chlamydia infections, cholera, clostridium infections, coccidioidomycosis, cryptococcosis, dermatomycoses, diphtheria, ehrlichiosis, epidemic louse borne typhus, Escherichia coli infections, fusobacterium infections, gangrene, general infections, general mycoses, gonorrhea, gram-negative bacterial infections, gram-positive bacterial infections, histoplasmosis, impetigo, klebsiella infections, legionellosis, leprosy, leptospirosis, listeria infections, lyme disease, malaria, maduromycosis, melioidosis, mycobacterium infections, mycoplasma infections, necrotizing fasciitis, nocardia infections, onychomycosis, ornithosis, pneumococcal infections, pneumonia, pseudomonas infections, Q fever, rat-bite fever, relapsing fever, rheumatic fever, rickettsia infections, Rocky-mountain spotted fever, salmonella infections, scarlet fever, scrub typhus, sepsis, sexually transmitted bacterial diseases, staphylococcal infections, streptococcal infections, surgical site infection, tetanus, tick-borne diseases, tuberculosis, tularemia, typhoid fever, urinary tract infection, vibrio infections, yaws, yersinia infections, Yersinia pestis plague, zoonoses and zygomycosis.

Medical conditions that are associated with fungi and are treatable by amine-borane compounds according to the present embodiments mainly include fungal infections or mycoses, as is detailed hereinunder.

Fungal infections or mycoses are classified depending on the degree of tissue involvement and mode of entry into the host. Main classes are superficial, subcutaneous, systemic and opportunistic infections.

Superficial mycoses infection is localized to the skin, the hair, and the nails. An example is “ringworm” or “tinea”, an infection of the skin by a dermatophyte. Ringworm refers to the characteristic central clearing that often occurs in dermatophyte infections of the skin. Dermatophyte members of the genera Trycophyton, Microsporum and Epidermophyton are responsible for the disease. Tinea can infect various sites of the body, including the scalp (tinea capitis), the beard (tinea barbae) the foot (tinea pedis: “athlete's foot”) and the groin (tinea cruris).

Candida albicans is a yeast causing candidiasis or “thrush” in humans. As superficial mycoses, candidiasis typically infects the mouth or vagina. C. albicans is part of the normal flora of the vagina and gastrointestinal tract and is therefore considered “commensal”.

Subcutaneous mycoses are infections confined to the dermis, subcutaneous tissue or adjacent structures. Infection may arise following the wounding of the skin and the introduction of vegetable matter. These mycoses are rare and confined mainly to tropical regions, and tend to be slow in onset but chronic in duration. An example is sporotrichosis caused by Sporothrix schenckii. The fungus is dimorphic, being a mould that can convert to a yeast form at 37° C. on rich laboratory media or in bodily infection. The disease is most prevalent the Americas, South Africa and Australia, but Sporotrichosis is also seen in Europe and other parts of the world. Infection usually follows and insect bite, a thorn prick or scratch from a fish spine. Certain occupation groups appear to have increased risk from infection. These include florists, farm workers and others who handle hay and moss. The most common symptom is an ulcerative lesion that may develop into lymphangitis.

Systemic mycoses divide into primary and opportunistic. These are invasive infections of the internal organs where the organism gains entry to the lungs, gastrointestinal tract or through intravenous lines. These infections may be caused either by primary pathogenic fungi or by opportunistic fungi that are of marginal pathogenicity but can infect the immunocompromised host.

Primary pathogenic fungi infection occurs in previously healthy persons and arises through the respiratory route. Examples include histoplasmosis, blastomycosis, coccidiomycosis and paracoccidiodomycosis. The fungi occur throughout the world.

Histoplasmosis is caused by Histoplasma capsulatum which is dimorphic (being a mould that can convert to a yeast form). It is found in the soil and growth is enhanced by the presence of bird and bat excreta. Environments containing such material are often implicated as sources of human infection. The lungs are the main site of infection but dissemination to the liver, heart and central nervous system can occur. Pulmonary infection can resemble symptoms seen in tuberculosis.

Opportunistic fungi may attack patients whom usually have some serious immune or metabolic defect, or have undergone surgery. The diseases include aspergillosis, systemic candidosis and cryptococcosis. Exceptionally, other fungi that are normally not pathogenic, such as Trichosporon, Fusarium or Penicillium, may cause systemic infections.

The term Aspergillosis collectively refers to a number of different diseases caused by the mould Aspergillus. It produces large numbers of spores and occurs world-wide. The organism can infect the lungs, inner ear, sinuses and, rarely, the eye of previously healthy persons. In the immunosuppressed host, Aspergillus can disseminate throughout the body.

Candidosis caused by, for example, C. albicans which is part of the normal human flora (see hereinabove), can proliferate and disseminate throughout the body in severely immunocompromised patients (e.g., those receiving chemotherapy).

Cryptococcosis is a systemic infection caused by the yeast Cryptococcus neoformans. The commonest manifestation is a subacute or chronic form of meningitis resulting from the inhalation of the organism. Pulmonary infection can also occur. The disease affects both healthy and immunosuppressed individuals and occurs world-wide. C. neoformans can be isolated in large numbers from pigeon droppings in the environment, although such birds do not appear to harbor the yeast.

Other fungal related diseases may occur as a result of constant exposure to fungal spores in the atmosphere, leading to respiratory allergies. Elevated antibodies to a range of common spore forming fungi have been demonstrated in occupational diseases such as humidifier fever, malt workers' lung and wheat threshers' disease.

Many moulds produce secondary metabolites (mycotoxins) that are highly toxic to humans. Ergotism is caused by eating bread prepared from rye infected with the fungus Claviceps purpurea. Historically, several large scale outbreaks of madness in local populations have been attributed to ergotism.

Pneumocystis is an infection of the lung caused by Pneumocystis carinii. The organism is a common cause of fatal pneumonia in AIDS patients. An intracellular parasite, with a life cycle of trophozoite and cyst, it was formerly considered to be a protozoan. However, comparisons of DNA and RNA sequences have established that it is one of the group of ustomycetous red yeast fungi. The cysts contain 8 nuclei which can be seen in smears of pulmonary aspirates. P. carinii is a commensal of many wild and domestic animals and evidence suggests that human infection is commonly derived from dogs.

Medical conditions associated with pathogenic parasites and protozoa which, according to the present embodiments, are treatable by amine-borane compounds include, without limitation, acanthamoeba infection, African trypanosomiasis (sleeping sickness), alveolar echinococcosis, amebiasis (entamoeba histolytica infection), American trypanosomiasis (Chaga's disease), ancylostoma infection (hookworm infection, cutaneous larva migrans, CLM), angiostrongylus infection (angiostrongyliasis), angiostrongyliasis (angiostrongylus infection), anisakis infection (anisakiasis), anisakiasis (anisakis infection), ascariasis (intestinal roundworms), babesia infection (babesiosis), babesiosis (babesia infection), balantidiasis (balantidium infection), balantidium infection (balantidiasis), baylisascaris infection (racoon roundworm), bilharzia (schistosomiasis), blastocystis hominis infection, body and public lice infestation (“the crabs”), capillaria infection (capillariasis), capillariasis (capillaria infection), cercarial dermatitis (swimmer's itch), chilomastix mesnili infection, clonorchis infection (clonorchiasis), clonorchiasis (clonorchis infection), cryptosporidiosis (cryptosporidium infection), cutaneous larva migrans (CKM, hookworm infection, ancylostoma infection), cyclospora infection (cyclosporiasis), cysticercosis (neurocysticercosis), delusional parasitosis, dientamoeba fragilis infection, diphyllobothrium infection (diphyllobothriasis), dipylidium infection (dog or cat tapeworm infection), dracunculiasis (guinea worm disease), dog tapeworm (dipylidium), E. histolytica infection (amebiasis) echinococcosis (alveolar hydatid disease), elephantiasis (filariasis, lymphatic filariasis), endolimax nana infection, Entamoeba coli infection, Entamoeba dispar infection, Entamoeba hartmanni infection, Entamoeba histolytica infection (amebiasis), Entamoeba polecki infection, Enterobiasis (pinworm infection), fasciola infection (fascioliasis), fascioliasis (fasciola infection), fasciolopsiasis (fasciolopsis infection), fasciolopsis infection (fasciolopsiasis), filariasis (lymphatic filariasis, elephantiasis), foodborne diseases, giardiasis (giardia infection, “beaver fever”), gnathostoma infection (gnathostomiasis), gnathostomiasis (gnathostoma infection), guinea worm disease (dracunculiasis), heterophyes infection (heterophyiasis), hymenolepis infection (hymenolepiasis), hookworm infection (ancylostoma infection, cutaneous larva migrans), intestinal roundworms (ascariasis), iodamoeba buetschlii infection, isospora infection (isosporiasis), leishmaniasis (kala-azar, leishmania infection), loa-loa infection (loaiasis), lymphatic filariasis (filariasis, elephantiasis), malaria, microsporidia infection (microsporidiosis), naegleria infection, neurocysticercosis (cysticercosis), nonpathogenic intestinal amebae infection, onchocerciasis (river blindness), opisthorchis infection (opisthorchiasis), paragonimus infection (paragonimiasis), pediculosis (head lice infestation), pinworm infection. (enterobiasis), Pneumocystis carinii pneumonia (PCP), raccoon roundworm infection (baylisascaris infection), river blindness (onchocerciasis), scabies (mite infestation), schistosomiasis (bilharzia), strongyloides infection (strongyloidiasis), swimmer's itch (cercarial dermatitis), taenia infection (tapeworm infection), tapeworm infection (taenia infection), toxocara infection (toxocariasis, ocular larva migrans, visceral larva migrans), toxocariasis (toxocara infection, ocular larva migrans, visceral larva migrans), toxoplasmosis (toxoplasma infection), trichinellosis (trichinosis), trichinosis (trichinellosis), trichomonas infection (trichomoniasis), trichomoniasis (trichomonas infection), trichuriasis (whipworm infection, trichuris infection), travelers' diarrhea, waterborne diseases and zoonotic diseases (diseases spread from animals to people).

Particularly preferred medical conditions that are treatable by amine-borane compounds according to the present embodiments include malaria and leishmania. As discussed hereinabove, no cure has been found hitherto to these fatal conditions. As mentioned hereinabove and is demonstrated in the Examples section that follows, amine-borane compounds were found highly active against exemplary malarial and leishmanial strains.

In each of the various aspects described herein, the amine-borane compounds can be used in combination with one or more other therapeutically active agents that are capable of treating a specified medical condition. Thus, amine-borane compounds can be used along with other anti-fungal, anti-malarial, anti-bacterial, anti-leishmanial and other antimicrobial agents.

In any of the above aspects of the present invention, the amine borane compounds can be utilized either per se or, preferably, as a part of a pharmaceutical composition that further comprises a pharmaceutically acceptable carrier.

Thus, according to additional aspects of the present invention, there are provided pharmaceutical compositions, which are identified for use in the treatment of any of the medical conditions listed above, and which comprise one or more amine-borane compounds and a pharmaceutically acceptable carrier.

As used herein a “pharmaceutical composition” refers to a preparation of amine-borane compounds, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present invention thus may be formulated in conventional manner using one or more pharmaceutically acceptable carriers comprising excipients and auxiliaries, which facilitate processing of the silver-coated enzymes into preparations which, can be used pharmaceutically. Proper formulation is dependent upon the route of administration chosen. The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

The pharmaceutical composition may be formulated for administration in either one or more of routes depending on whether local or systemic treatment or administration is of choice, and on the area to be treated. Administration may be done topically (including ophtalmically, vaginally, rectally, intranasally), orally, by inhalation, or parenterally, for example by intravenous drip or intraperitoneal, subcutaneous, intramuscular or intravenous injection.

Formulations for topical administration may include but are not limited to lotions, ointments, gels, creams, suppositories, drops, liquids, sprays and powders. Conventional pharmaceutical carriers, aqueous, powder or oily bases, thickeners and the like may be necessary or desirable.

Compositions for oral administration include powders or granules, suspensions or solutions in water or non-aqueous media, sachets, capsules or tablets. Thickeners, diluents, flavorings, dispersing aids, emulsifiers or binders may be desirable.

Formulations for parenteral administration may include, but are not limited to, sterile solutions which may also contain buffers, diluents and other suitable additives. Slow release compositions are envisaged for treatment.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a silver-coated enzyme of the invention formulated in a compatible pharmaceutical carrier may also be prepared, placed in an appropriate container, and labeled for treatment of an indicated condition or diagnosis, as is detailed hereinabove.

Thus, according to an embodiment of the present invention, depending on the selected amine-borane compound and the presence of additional active ingredients, the pharmaceutical compositions of the present invention are packaged in a packaging material and identified in print, in or on the packaging material, for use in the treatment of a medical condition associated with a pathogenic microorganism, as is defined hereinabove.

Microorganisms such as fungi, bacteria and protozoa, are firmly and directly involved in human life. Some fungi are parasitic, and cause devastating plant infections; though only about 50 species are known to harm animals. Serious agricultural pests, parasitic fungi such as the “rusts” and the “smuts” can blight entire crops, especially affecting cereals such as wheat and corn. Microbial life forms such as fungi and bacteria may flourish on any organic matter, using it as a food source either aerobically or anaerobically. In more instances than not, this growth is damaging and undesired, or at least required to be under control (e.g., fermentation of sugars, milk and the like).

Therefore, in addition to the above-described uses of amine-borane compounds as therapeutic agents for treating various medical conditions, the antimicrobial activity of amine-borane compounds described herein can be utilized in many industries and agricultural branches where application of an antimicrobial agent is beneficial. These include, for example, application to, for example, various plant and tree parts (fruits, leaves, roots, seeds); industrial products such as food products, cosmetics, and paints; live-stock, fisheries, hatcheries, egg and poultry, beehives and the like, and for treatment of various vessels, surfaces and constructs which are routinely treated by anti-microbial and antifungal agents.

Thus, according to a further aspect of the present invention there is provided a method of reducing the load of a pathogenic, or otherwise undesired or acceptable microorganism in a substrate, the method includes applying to the substrate an antimicrobial effective amount of an amine-borane compound.

The term “reducing the load” refers to a decrease in the number of the microorganism(s), or to a decrease in the rate of their growth or both in the substrate as compared to a non-treated substrate.

The term “substrate” as used herein, refers to any surface, structure, product or material which can support, harbor or promote the growth of a microorganism. Non-limiting examples include the inner walls of a storage container that is routinely treated with anti-microbial preferably anti-fungal agents, a soil and/or soil enrichment supplements, any agricultural product or crop such as wood, fiber, fruit, vegetable, flower, extract, horticultural crop and any other processed or unprocessed agricultural product or crop which are produced from organic origins such living plants or animals, a cosmetic product, a building, warehouse, compartment, container or transport vehicle, a dye or a paint and any other materials and industrial compounds used for which require protection of their surfaces against microbes, moulds and fungi attacks, such as, for example, construction materials.

As used in the context of this aspect, the phrase “antimicrobial effective amount” describes an amount of the compound which will reduce to some extent the population of the microorganisms in a substrate harboring the microorganism.

Examples of soil, product, material and structure infecting microorganisms include any soil-borne plant pathogenic fingi, plant pathogenic bacteria, wood decay fungi and plant pathogenic nematodes. Soil-borne pathogenic fungi include, but are not limited to, Cylindrocarpom spp., Fusarium spp., Phoma spp., Phytophtora spp., Pythium spp., Rhizoctonia spp., Sclerotinia spp., Verticillium spp. and Macrophomina spp. Soil-borne plant pathogenic bacteria include, but are not limited to Pseudomonas spp., Xanthomonas spp., Agrobacterium tumefaciense, Corynobacterium spp. and Streptomycess spp. Plant pathogenic nematodes include, but not limited to, Meloidogyne spp., Xiphinema spp., Pratylenchus spp., Longidorus spp., Paratylenchus spp., Rotylenchulus spp., Helicotylenchus spp., Hoplolaimus spp., Paratrichodorus spp., Tylenchorhynchus spp., Radopholus spp., Anguina spp., Aphelenchoides spp., Bursapehlenchus spp., Ditylenchus spp., Trichchodorus spp., Globodera spp., Hemicycliophora spp., Heterodera spp., Dolichodorus spp., Criconemoides spp., Belonolaimus spp. and Tylenchulus semipenetrans.

As discussed hereinabove, the amine borane presented herein can also be used to reduce the load of a resistant microorganism in a substrate. Similarly to drug-resistance, resistance can be manifested also with respect to non-drug antimicrobial agents such as pesticides and the likes. The resistant microorganism is one that is not susceptible to conventionally used antimicrobial agents. Hence, preferably the pathogenic or otherwise undesired or acceptable microorganism is a resistant microorganism.

The present invention further relates to a wide range of products and materials comprising a substrate or a product, and an amino-borane as an antimicrobial agent.

Thus, according to an additional aspect of the present invention there is provided an article-of-manufacturing which includes a product and an anti-microbial effective amount of an amine-borane compound, as described herein.

Such products include, for example, food products, agricultural products, cosmetic products and many more. Due to its effect in reducing the load of microorganisms, an amine-borane compound can be utilized as a preservative in such products.

As is demonstrated in the Examples section that follows, a variety of structurally diverse amine-borane compounds were tested and found highly active against a wide spectrum of microbial strains. These include, for example, alkylamino cyanoboranes having a variable alkyl chain (e.g., short, long, saturated and unsaturated), halogenated aminoboranes, amino bis-boranes and more.

Thus, preferred amine-borane compounds that can be beneficially utilized in any of the above aspects of the present invention are collectively represented by Formula I and Formula II, as follows:

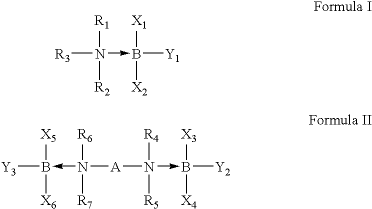

wherein Y₁, Y₂ and Y₃ are each independently selected from the group consisting of a cyano group (—C≡N), a —C(═O)Ra group, amine and alkyl, whereas Ra is selected from the group consisting of hydrogen, halo, hydroxy, alkoxy, thiohydroxy, thioalkoxy, aryloxy, thioaryloxy and amine; X₁-X₆ are each independently selected from the group consisting of hydrogen, alkyl, halo, cycloalkyl, and aryl; R₁-R₇ are each independently selected from the group consisting of hydrogen, alkyl, alkenyl, alkynyl, cycloalkyl and aryl or, alternatively, two of R₁-R₃, R₄ and R₅ and/or R₆ and R₇ form a carbocyclic ring (e.g., a cycloalkyl or aryl); and A is a substituted or non-substituted, saturated or non-saturated hydrocarbon having from 1 to 20 carbon atoms.

As used herein, the term “hydroxy” describes an —OH group.

As used herein, the term “halo” describes fluoro, chloro, bromo or iodo.

The term “alkoxy” describes a —OR group, where R is alkyl, cycloalkyl, aryl, as these terms are defined herein.

The term “thiohydroxy” and/or “thiol” refers to a —SH group.

The term “thioalkoxy” describes a —SR group, where R is as defined herein.

The term “aryloxy” describes both an —O-aryl and an —O-heteroaryl group, as defined herein.

The term “thioaryloxy” describes both an —S-aryl and an —S-heteroaryl group, as defined herein.

As used herein, the term “amine” describes a —NR′R″ group where each of R′ and R″ is independently hydrogen, alkyl, cycloalkyl or aryl, as these terms are defined herein.

As used herein, the term “alkyl” describes an aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. The alkyl can be substituted or unsubstituted. The alkyl can further be saturated or unsaturated.

An unsaturated alkyl is also referred to herein as alkenyl or alkynyl, as these terms are defined herein.

The term “alkenyl” describes an unsaturated alkyl having at least two carbon atoms and at least one carbon-carbon double bond.

The term “alkynyl” an unsaturated alkyl having at least two carbon atoms and at least one carbon-carbon triple bond.

The term “cycloalkyl” describes an all-carbon monocyclic or fused ring (i.e., rings which share an adjacent pair of carbon atoms) group where one or more of the rings does not have a completely conjugated pi-electron system.

The term “aryl” describes an all-carbon monocyclic or fused-ring polycyclic (i.e., rings which share adjacent pairs of carbon atoms) groups having a completely conjugated pi-electron system.

According to preferred embodiments of the present invention, the amine borane compounds utilized in the various aspects of the present invention are amine cyanoboranes and/or amine carboxyboranes.

Thus, preferably, each of Y₁, Y₂ and Y₃ in Formulae I and II above is independently selected from the group consisting of a cyano group (—C≡N), and a carboxy (—C(═O)Ra) group, wherein Ra is selected from the group consisting of hydrogen, halo, hydroxy, alkoxy, thiohydroxy, thioalkoxy, aryloxy, thioaryloxy, thiol and amine. The carboxy group can thus be a carboxylic acid (where Ra is hydroxy), or a derivative thereof such as, for example, an aldehyde (where Ra is hydrogen), an acyl halide (where Ra is halo), an ester (where Ra is alkoxy or aryloxy), a thioester (where Ra is thioalkoxy or thioaryloxy) or an amide (where Ra is amine).

Preferred amino carboxyboranes according to the present embodiments include esters derivatives, where Ra in the Formulae above is alkoxy. More preferably, the alkoxy is methoxy or ethoxy.

Further preferred compounds according to the present embodiments include alkylated aminoboranes, such that one or more of R₁-R₇ in Formulae I and II above is alkyl.

In one embodiment of the present invention, the alkyl is a short, saturated alkyl having 1-10 carbon atoms, preferably 1-8 carbon atoms, more preferably 1-6 carbon atoms and more preferably 1-4 carbon atoms. Representative examples include methyl, ethyl and n-butyl.

In still another embodiment, one or more of R₁-R₇ in Formulae I and II above is an unsaturated moiety such alkenyl or alkynyl. As is further demonstrated in the Examples section that follows, the incorporation of such an unsaturated moiety results in a significant increase of the anti-microbial activity of the compounds.

In U.S. Provisional Patent Application No. 60/716,082, filed Sep. 13, 2005, and in a PCT International Patent Application, by the present assignee, having Attorney Docket No. 32587, which is entitled “novel amine-borane compounds and uses thereof” and is co-filed on the same date as the instant application, both are incorporated by reference as if fully set forth herein, novel amine-borane compounds having a long alkyl substituent are disclosed. These compounds were found to exhibit high anti-microbial activity, as is demonstrated hereinafter.

Thus, in another embodiment, one or more of R₁—R₇ is a long (high) alkyl, having more than 10 carbon atoms, preferably 11-17 carbon atoms, and more preferably 11-15 carbon atoms. As demonstrated in the Examples section that follows, amino borane compounds having a C₁₋₅ alkyl substituent were found to exert superior anti-microbial activity as compared to structurally similar compounds having a longer or shorter alkyl substituent.

In an additional embodiment, the long alkyl is unsaturated and thus is a long alkenyl or alkynyl.

Representative examples of amine-borane compounds having the general Formula I and a high (long) alkyl substituent that are particularly suitable for use in the context of the present invention include, without limitation, pentyl-dimethyl-amine cyanoborane, dimethyl-undecyl-amine cyanoborane, dimethyl-undecyl-amine cyanobromoborane, dimethyl-undecyl-amine cyanodibromoborane, dodecyl-dimethyl-amine cyanoborane, 1-dimethylamino-2-methyl-octan-2-ol cyanoborane, dimethyl-nonyl-amine cyanoborane, dimethyl-tridecyl-amine cyanoborane, dimethyl-pentadecyl-amine cyanoborane, heptadecyl-dimethyl-amine cyanoborane, 1-dimethylamino-dodecan-2-ol cyanoborane, dimethyl-undecyl-amine cyanofluoroborane and 1-dimethylamino-undecan-2-ol cyanoborane.

In yet another embodiment, each of X₁-X₅ is independently hydrogen or halo. In one embodiment, at least one of X₁ and X₂ in Formula I and/or at least one of X₃-X₆ in Formula II is halo.

The halo substituent can be, for example, bromo. As is demonstrated in the Examples section that follows, the incorporation of a bromo substituent did not affect the activity of the amino borane compounds. The incorporation of two bromo substituents resulted in diminished activity yet these compounds exhibited a reasonable activity.

In the above mentioned U.S. Provisional Patent Application No. 60/716,082 and PCT International Patent Application, by the present assignee, having Attorney Docket No. 32587, novel fluorinated aminoborane compounds, in which one or more of X₁-X₆ is fluoro are also disclosed. As is demonstrated in the Examples section that follows, these compounds were found highly active against various microbial strains, and showed comparable activity to brominated analogs thereof.

Thus, preferably, the halo substituent is fluoro. Representative examples of such fluorinated aminoboranes that are particularly suitable for use in the context of the present invention include, without limitation, dimethyl-undecyl-amine cyanofluorobromoborane, trimethyl-amine cyanofluoroborane, ethyl-dimethyl-amine cyanofluoroborane, butyl-dimethyl-amine cyanofluoroborane, trimethyl-amine carboxyfluoroborane methyl ester, trimethyl-amine carboxyfluoroborane ethyl ester, ethyl-dimethyl-amine carboxyfluoroborane methyl ester, butyl-dimethyl-amine carboxyfluoroborane methyl ester, trimethyl-amine cyanodifluoroborane, trimethyl-amine carboxydifluoroborane methyl ester, trimethyl-amine carboxydifluoroborane ethyl ester, trimethyl-amine cyanofluorobromoborane, trimethyl-amine carboxyfluorobromoborane ethyl ester, triethyl-amine carboxydifluoroborane, dimethyl-undecyl-amine cyanofluoroborane and (2-fluoro-nonyl)-dimethyl-amine cyanoborane.

Additional representative examples of amine-borane compounds which are suitable for use in the context of the present invention include, without limitation, amine-borane compound having the general Formula I above such as 1-dimethylaminomethyl-cyclopent-2-enol cyanoborane, (2-hydroxy-2-phenyl-ethyl)-dimethyl-amine cyanoborane, ethyl-dimethyl-amine cyanoborane, but-3-enyl-dimethyl-amine cyanoborane, trimethyl-amine cyanodibromoborane, trimethyl-amine cyanoborane, butyl-dimethyl-amine cyanoborane and dimethyl-trimethylsilanylmethyl-amine cyanoborane.

As described hereinabove, additional amine-borane compounds that are suitable for use in the context of the present invention are amino bis-borane compounds such as those having the general Formula II.

As shown in Formula II above, such amino bis-borane compounds include two aminoborane moieties that are linked therebetween by a hydrocarbon moiety (denoted as A in Formula II). The hydrocarbon moiety can be saturated or unsaturated, linear or branched, substituted or unsubstituted and preferably has from 1 to 20 carbon atoms.

In a preferred embodiment of the present invention, the hydrocarbon is a saturated, unsubstituted hydrocarbon having from 5 to 20 carbon atoms. Preferably, the hydrocarbon has 10-20 carbon atoms and more preferably 10-14 carbon atoms. Amino bis-borane compounds having such a hydrocarbon are also disclosed in the above mentioned U.S. Provisional Patent Application No. 60/716,082 and in a PCT International Patent Application, by the present assignee, having Attorney Docket No. 32587.

Thus, representative examples of amine bis-borane compounds having the general Formula II, that are suitable for use in the context of the present invention include, without limitation, N,N,N′,N′-tetramethyl-decane-1,10-diamine cyanoborane, N,N,N′,N′-tetramethyl-decane-1,10-diamine bis-cyanobromoborane, N,N,N′,N′-tetramethyl-decane-1,10-diamine bis-cyanodibromoborane, N,N,N′,N′-tetramethyl-decane-1,10-diamine bis-carboxyborane, N,N,N′,N′-tetramethyl-dodecane-1,12-diamine bis-cyanoborane and N,N,N′,N′-tetramethyl-tetradecane-1,14-diamine bis-cyanoborane.

While reducing the present invention to practice and testing the compounds presented herein for antimicrobial activity, it was generally observed, as presented in the Examples section that follows, that the efficacy of the compounds is strongly linked to the length of the N-alkyl chain in one or more of R₁-R₃ or R₄-R₇ of the compounds having the general Formulae I and II. The longer the alkyl chain, the higher bioactivity activity the compounds exhibited, as demonstrated for Compound K-R (see, Table 1 below). Therefore, the preferred chain length for one or more of R₁-R₃ or R₄-R₇ the compounds having the general Formulae I and II, according to the present invention, and particularly to one or more of R₁-R₃ in Formula I, is between eleven and fifteen carbon-long chain, and more preferred is a fifteen long (C₁₅) chain.

It was further found that the compounds having a longer N-alkyl-N chain exhibited higher bioactivity activity. Therefore, the preferred length of A in Formula II (the N-alkyl-N chain), according to the present invention, is a fourteen carbon-long chain (C₁₄).

It was further found that the presence of an unsaturated C═C double bond at of the terminal bond of the alkyl group of either of R₁-R₃ or R₄-R₇ of the compounds having the general Formulae I and II, and particularly in one or more of R₁-R₃ in Formula I, dramatically enhanced the biologic activity thereof, as demonstrated in the Examples section that follows for Compound K-D and Compound K-V.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Materials and Methods

The following amine-boranes were prepared according to published procedures: (2-hydroxy-2-phenyl-ethyl)-dimethyl-amine cyanoborane (K-B) [15], ethyl-dimethyl-amine cyanoborane (K-C) [17], but-3-enyl-dimethyl-amine cyanoborane (K-D) [17], trimethyl-amine cyanodibromoborane (K-E) [Takrouri et al., J. Organometallic Chem. y, 2005, 690, 4150; Györi et al., Inorg. Chim. Acta, 1994, 218, 21], trimethyl-amine cyanoborane (K-F) [Wisian-Neilson et al., Inorg. Chem. 1978, 17, 2327], butyl-dimethyl-amine cyanoborane (K-G) [17], pentyl-dimethyl-amine cyanoborane (K-H) [17] and dimethyl-trimethylsilanylmethyl-amine cyanoborane (K-K) [17].

The following amine-boranes were prepared according to the procedure described in a U.S. Provisional Patent Application No. 60/716,082 and in a PCT International Patent Application, by the present assignee, having Attorney Docket No. 32587, which is co-filed on the same date as the instant application: 1-dimethylaminomethyl-cyclopent-2-enol cyanoborane (K-A), dimethyl-undecyl-amine cyanoborane (K-I), dimethyl-undecyl-amine cyanobromoborane (K-I₁), dimethyl-undecyl-amine cyanodibromoborane (K-I₂), dimethyl-undecyl-amine cyanofluorobromoborane (K-I₃), dimethyl-undecyl-amine cyanofluoroborane (K-I₄), N,N,N′,N′-tetramethyl-decane-1,10-diamine cyanoborane (K-J), N,N,N′,N′-tetramethyl-decane-1,10-diamine bis-cyanobromoborane (K-J₁), N,N,N′,N′-tetramethyl-decane-1,10-diamine bis-cyanodibromoborane (K-J₂), N,N,N′,N′-tetramethyl-decane-1,10-diamine bis-carboxyborane (K-J₃), dodecyl-dimethyl-amine cyanoborane (K-L), N,N,N′,N′-tetramethyl-dodecane-1,12-diamine bis-cyanoborane (K-M), N,N,N′,N′-tetramethyl-tetradecane-1,14-diamine bis-cyanoborane (K-N), 1-dimethylamino-2-methyl-octan-2-ol cyanoborane (K-O), dimethyl-nonyl-amine cyanoborane (K-P), dimethyl-tridecyl-amine cyanoborane (K-Q), dimethyl-pentadecyl-amine cyanoborane (K-R), heptadecyl-dimethyl-amine cyanoborane (K-S), 1-dimethylamino-dodecan-2-ol cyanoborane (K-T), hex-5-enyl-dimethylamine cyanoborane (K-V), 1-dimethylamino-undecan-2-ol cyanoborane (K-U), trimethyl-amine cyanofluoroborane (Compound 1), ethyl-dimethyl-amine cyanofluoroborane (Compound 2), butyl-dimethyl-amine cyanofluoroborane (Compound 3), trimethyl-amine carboxyfluoroborane methyl ester (Compound 4); trimethyl-amine carboxyfluoroborane ethyl ester (Compound 5), ethyl-dimethyl-amine carboxyfluoroborane methyl ester (Compound 6), butyl-dimethyl-amine carboxyfluoroborane methyl ester (Compound 7), trimethyl-amine cyanodifluoroborane (Compound 8), trimethyl-amine carboxydifluoroborane methyl ester (Compound 9), trimethyl-amine carboxydifluoroborane ethyl ester (Compound 10), trimethyl-amine cyanofluorobromoborane (Compound 11), trimethyl-amine carboxyfluorobromoborane ethyl ester (Compound 12) and triethyl-amine carboxydifluoroborane (Compound 13).

### Example 1

### Antifungal Activity Assays

Antifungal activity of the amine borane compounds was determined using in-vitro susceptibility tests by microbroth dilution method, as detailed below.

Several yeast strains were used for susceptibility testing, namely Candida albicans CBS 562 (a reference type strain of the main common cause of yeast infection in humans; Centraalbureau voor Schimmelcultures, Utrecht, The Netherlands) and two clinical isolates, 607, and 615; Candida glabrata 4210; Candida glabrata 4475, a yeast strain is known to be a resistant to azoles; Candida glabrata 566, 572, 578, 646, and 648 and Candida krusei 603 and 638; Candida glabrata 4681; Candida glabrata 4787; Saprolegnia parasitica T1, a water mould which causes a severe disease in tropical fish—Saprolegniasis and massive economical damages, with no effective treatment to date as well as the mold Aspergillus fumigatus ATCC 64026 (a reference strain; The American Type Culture Collection, Manassas, Va.), a mould which is the main cause of mould infection in immunocompromised host also a main cause of fungal infection in fowl. In addition, all isolates were obtained from disseminated candidemia patients from Hadassah-Hebrew University Medical Center (Isolate numbers refer to an internal index).

The in vitro susceptibility of the tested strains to each of the tested compounds was determined by the broth micro dilution method according to National Committee for Clinical Laboratory Standards for yeasts (NCCLS/CLSI) recommendations for yeasts (M27-A241) [10] and for filamentous fungi (M-38A42) [27]. Briefly, 2-fold serial dilutions of drugs from stock solutions were prepared in an RPMI-1640 broth medium (Sigma, St. Louis, Mo.) buffered to a final pH of 7.0 with 0.165 M morpholinepropanesulfonic acid (MOPS; Sigma) and 1M NaOH, and sterilized by filtration and inoculated with 10⁴ cells per ml. A stock solution of 10 mg/ml was prepared in dimethyl sulfoxide (DMSO, Sigma) for the various amine cyanoborane compounds and for two conventional anti-fungal agents, amphotericin B (referred to herein as CAF1) and fluconazole (referred to herein as CAF2), which were used as controls. The final drug concentrations in the test ranged from 1024 to 4 mg/L in a final volume of 0.1 ml.

The minimum fungicidal concentration (MFC) was established as the lowest concentration of drug producing negative subcultures, after plating 20 μl of each clear well after 72 hours incubation at 35° C. on drug-free SDA plate.

Specifically, fungal inocula were prepared from 24-hours (for Candida sp.) or 72 hours (for A. fumigatus) cultures on SDA plates (Difco, Detroit, Mich.). The inocula were harvested by harvesting a single colony of yeast into a sterile saline tube. Mold cultures were suspended in plates with sterile saline containing a 0.05% (v/v) Tween-20 (Difco) suspension and pipeted into sterile tubes and allowed to rest for 30 minutes for the debris to sink down. The supernatant was then transferred to a fresh tube. Both yeast and mold were diluted into RPMI-1640 broth medium to yield a final inoculum concentration of 2×10³ yeast per ml for Candida and 2×10⁴ spores per ml for Aspergillus, as measured by counting the initial suspension with a hemacytometer. The micro dilution wells, which contained 0.1 ml of the serially diluted drug, were inoculated with 0.1 ml of the resulting suspension. The final inoculum concentration after dilution with the drug suspension was 10³-10⁴ cells per ml. Two wells containing the drug-free medium and inoculum were used as controls. The inoculated plates were incubated at 35° C. for 24 hour (for Candida sp.) or 72 hours (for A. fumigatus). The growth in each well was then visually estimated. The MICs were determined visually, and were defined as the lowest drug concentration at which there was complete absence of growth (MIC-0).

The results of the antifungal activity of amine-borane compounds are presented in Table 1 below, wherein the MIC values of various compounds against various fungi are given in μmol/L or mg/L.

Column A of Table 1 presents the antifungal activity against Candida albicans CBS 562;

Column B of Table 1 presents the antifungal activity against Candida albicans 607;

Column C of Table 1 presents the antifungal activity against Candida albicans 615;

Column D of Table 1 presents the antifungal activity against Candida glabrata 4210;

Column E of Table 1 presents the antifungal activity against Candida glabrata 4475 (resistant to azoles);

Column F of Table 1 presents the antifungal activity against Candida glabrata 4681;

Column G of Table 1 presents the antifungal activity against Candida glabrata 4787;

Column H of Table 1 presents the antifungal activity against Saprolegnia parasitica T1; and

Column I of Table 1 presents the antifungal activity against Aspergillus fumigatus 64026.

As can be seen in Table 2, the amine cyanoboranes with diverse structures exhibited considerable antifungal activity. Particularly significant activity was demonstrated with amine cyanoborane compounds such as, for example, Compounds K-I, K-I₂ and K-N (see, entries 9, 11 and 20 respectively in Table 1 below).

Structure Activity Relationship (SAR) Studies:

A number of relationships were observed regarding the effect of the structure of the amine cyanoboranes and carboxyboranes and their derivatives, according to the present invention, on the antifungal activity.

Effect of length of the N-alkyl chain in alkyldimethylamine cyanoboranes (see, Formula III above):

Compound K-F, Compound K-C, Compound K-G, Compound K-H, Compound K-P, Compound K-L, Compound K-Q, and Compound K-R (see, corresponding entries 6, 5, 7, 8, 22, 18, 23 and 24 in Table 2), have the general structure of RₙNMe₂-BH₂C≡N, with varying lengths of the N-alkyl chain (n) in the R═(CH₂)ₙ group.

As can be seen in Table 2, compounds having a longer N-alkyl chain exhibited higher antifungal activity. The optimized chain length was for Compound K-R(C₁₅ in the N-alkyl group). The chain length was restricted to this length because attempts to use longer chain lengths resulted in solubility problems, as in Compound K-S, which has a C₁₇ in the N-alkyl group.

Effect of length of the N-alkyl-N chain in diamine bis-cyanoboranes for the diamine cyanoboranes (see, Formula II above):

Compounds K-J, K-M, and K-N (see, corresponding entries 13, 19 and 20 in Table 2), have the general structure N≡CBH₂Me₂N—(CH₂)ₙ—NMe₂—BH₂C≡N, with varying N-alkyl-N chain length (n).

As can further be seen in Table 2, compounds having a longer N-alkyl-N chain exhibited higher antifungal activity, until a limit of insolubility is reached. The optimized chain length is exhibited in Compound K-N(C₁₄ in the N-alkyl group) as attempts to form compounds with longer chain lengths (C₁₆ and above, data not shown) resulted in solubility problems.

As can be seen in Table 2, bromination of the compounds of the present invention generally enhances the antifungal activity. The bromination of Compound K-F to give the dibromo derivative Compound K-E enhanced the activity against C. albicans CBS 562 from 5100 to 1970 μmol/L. Also, the bromination of Compound K-J to the monobromo derivative Compound K-J₁ and the dibromo derivative Compound K-J₂ enhanced the activity against C. albicans CBS 562 from 408 μmol/L for Compound K-J to 140 and 104 μmol/L for Compound K-J₁ and Compound K-J₂, respectively, whereas the bromination or the fluorination of Compound K-I to give Compound K-I₁, Compound K-I₂, and Compound K-I₄ had no such effect. It was found that bromination enhances the antifungal activity to a larger extent as compared with fluorination thereof.

As can further be seen in Table 2, the presence of unsaturated C═C double bond in the alkyl group dramatically enhanced the activity as seen in Compound K-G and Compound K-D, where the MIC value against C. albicans CBS 562 was reduced from 3570 to 470 μmol/L in the presence of a C═C double bond at the end of the alkyl group. For Compound K-H and Compound K-V, the effect on activity with increased N-alkyl chain length was even greater, taking into account the difference of one methylene (CH₂) unit.

As can further be seen in Table 2, other structural modifications of amine cyanoboranes, such as, for example, addition of a hydroxyl group in one of R₈—R₁₀ in General Formula III as in Compound K-O, Compound K-U, Compound K-T, Compound K-A and Compound K-R, an aromatic group in one of R₈-R₁₀ as in Compound K-B, an unsaturated cyclic group as in Compound K-A, and a silyl group as in Compound K-K, did not result in significant enhancement in the antifungal activity of the compounds.

Effect of conversion of the amine cyanoborane to the amine carboxyborane derivative enhanced the antifungal activity

As can be seen in Table 2 below the antifungal activity against C. albicans CBS 562 was enhanced from 408 μmol/L for Compound K-J (see, entry 13 in Table 2) to 188 μmol/L for its carboxyborane derivative Compound K-J₃ (see, entry 16 in Table 2).

As can further be seen in Table 2, the amine cyanoboranes and derivatives thereof possess activity against Candida glabrata 4475, known to be resistant to azoles (see, column 6 in Table 2 below).

As can be concluded from Table 2 below, the most promising compound at the present is dimethyl-undecyl-amine cyanoborane (Compound K-I) which was also tested against moulds, typically more resistant to antifungal agents, and exhibited dramatic activity against both Saprolegnia and Aspergillus. Furthermore, Compounds K-I, K-I₁ and K-I₃ showed little activity against Rhizopus oryzae with MIC of 63 μg/ml.

Comparisons of Antifungal Activity to that of Conventional Antifungal Agents Against Resistant Strains:

As is further seen in Table 1 below, the MIC values obtained for the novel amine-boranes described herein against the tested fungal strains were compared to those obtained for conventional antifungal drugs amphotericin B and fluconazole. Since these strains are not resistant to these drugs, the susceptibility of fungal strains which are known to be resistant to fluconazole was therefore tested, using the protocol described hereinabove.

Table 2 below summarizes the results of the anti-fungal assays conducted for the presently leading amine cyanoborane Compounds K-I and K-I₂, as they are reflected by the MIC values of Compounds K-I and K-I₂ against various fungal strains and particularly fungal strains that are resistant to fluconazole, given in μmol/L or μg/ml and compared to two conventional anti-fungal agents amphotericin B (CAF1) and fluconazole CAF2).

As can be seen in Table 2, Compound K-I is highly active against all fungi. As can further be seen in Table 2, isolates of C. glabrata, which generate considerably high fluconazole MICs, and isolates of C. krusei, which are known as intrinsically resistant to fluconazole, were highly susceptible to the lead Compound K-I and its dibromo derivative Compound K-I₂, exhibiting much lower MIC values than those obtained with fluconazole. These results strongly suggest that these compounds can become potent antifungal agents, particularly in cases where conventional drugs are not.

TABLE 2

| | MIC (μmol/L) | | | |
|---|---|---|---|---|
| Fungal Strain | K-I | K-I₂ | CAF1 | CAF2 |
| Candida globrata 566 | 32.5 | 20.2 | 0.13 | 26.1 |
| Candida globrata 572 | 32.5 | 40.4 | 1.07 | >836 |
| Candida globrata 578 | 32.5 | 40.4 | 2.14 | >836 |
| Candida globrata 646 | 32.5 | 20.2 | 0.41 | 52.2 |
| Candida globrata 648 | 32.5 | 20.2 | 0.82 | 26.1 |
| Candida krusei 603 | 16.25 | 10.05 | 4.32 | 104.5 ^{a} |
| Candida krusei 638 | 16.25 | 10.05 | 4.32 | 104.5 ^{a} |
| Candida albicans 563 | 2-4 ^{b} | | | |
| Cryptococcus neoformans H-99 | 0.4 ^{b} | | | |
| Cryptococcus neoformans B-3501 | 0.8 ^{b} | | | |
| Saprolegnia parasitica T-1 | 2-4 ^{b} | | | |
| Saprolegnia parasitica CBS 540.67 | 2-4 ^{b} | | | |

^{a} Candida krusei is intrinsically resistant to fluconazole;
^{b} Values given in μg/ml.

### Example 2

### Anti Leishmanial Activity Assays

The compounds presented herein were further tested for anti lishmenial activity (against Leishmania, the parasitic flagellated protozoan discussed hereinabove which causes diseases in animals and humans). To this end, various strains of leishmania were grown under optimal conditions, either in Schneiders or in RPMI 1640 medium (Bet-Haemek, Israel), containing 20% fetal calf serum. Some strains were isolated from humans infected with L. tropica, L. major and L. infantum. Some strains, including L. donovani, were received from the World Health Organization Leishmania Strain Bank which is located in the Kuvin Centre, in the Hebrew University of Jerusalem. These strains depict CL and VL leishmaniasis. Promastigotes and axenic amastigotes were grown as published [20].

The in vitro antilishmenial effect of Compound K-I, an exemplary amine borane compound of the present invention, was determined on promastigotes (the flagellate stage of the Leishmania parasites). Parasites, after 3 or 4 days of growth, were washed and resuspended in fresh medium containing fetal calf serum to a concentration of 15×10⁴ promastigotes in 200 μl and distributed into flat bottom wells. The plates were incubated at 28° C. in a wet chamber in an atmosphere of 5% CO₂, 5% O₂, and 90% N₂. After 24 hours, 0.5 μCi of [³H]thymidine was added to each well, the parasites were incubated for additional 24 hours, and then harvested on glass-microfiber filters. Radioactivity was measured in a liquid scintillation counter. Each treatment was performed in triplicate; the results are expressed as percent growth inhibition. [(100-cpm of wells with drug)/cpm of untreated wells]×100 (cpm stands for counts per minute), representing the MIC value in μg/ml [Golenser, J. et al., 1999, Antimicrob. Agents Chemother., 43:2209-2214].

The in vitro antilishmenial effect of Compound K-I was further tested on amastigotes (an intracytoplasmic, nonflagellated form of the Leishmania parasites). Peritoneal macrophages were obtained from 10-week-old to 12-week-old outbred mice previously stimulated for 4 or 5 days with 3% thioglycolate. The macrophages were distributed into 8-well slides to 2.5×10⁵ cells in 300 μl per well and incubated at 37° C. and 5% CO₂ for 3 hours in order to allow for cell adhesion. The supernatant was discarded, and promastigotes (8×10⁵ to 10×10⁵ per well) were added. After incubation overnight, fresh medium containing Compound K-I was added for an additional 24 to 48 hours. At the end of this period, the wells were washed, fixed with methanol, and stained with Giemsa (CAS No.: 51811-82-6). Each treatment was done in duplicate wells; 100 macrophages in each well and the number of amastigotes they contained were counted, representing the MIC value in μg/ml.

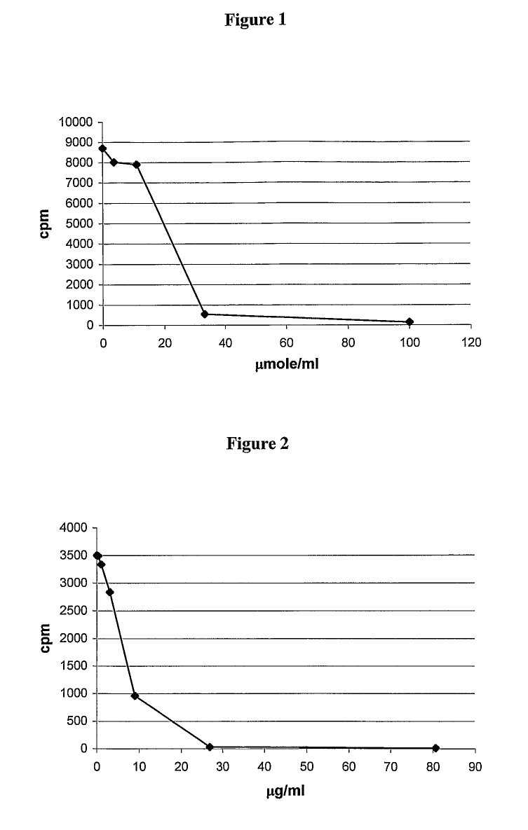

The results obtained with Compound K-I are presented in Table 3 below and further in FIG. 1. Table 3 presents the radioactivity, given in counts per minute (cpm), recorded for a parasite sample for each of the tested doses (given in μM/ml). A clear dose-dependent effect is seen. As can be seen in FIG. 1, the measured radioactivity, which is indicative of the live parasite, diminishes rapidly as a dose of the amine borane increases, indicating a strong and dose-dependent anti lishmenial effect.

TABLE 3

| μM/ml | cpm |
|---|---|
| 0 | 8699 |
| 3.6 | 8003 |
| 11 | 7898 |
| 33 | 537 |
| 100 | 138 |

### Example 3

Anti-Malarial Activity Assays

The compounds presented herein were further tested for anti lishmenial activity (against P. falciparum, the parasitic protozoan discussed hereinabove which causes malaria). To this end, cultures of P. falciparum were grown in human erythrocytes by standard methods under a low-oxygen atmosphere. The culture medium was RPMI 1640, supplemented with 40 mM HEPES, 25 mg/liter gentamicin sulfate, 10 mM D-glucose, 2 mM glutamine and 8% heat-inactivated plasma.

P. falciparum growth was assessed by measuring the incorporation of the radiolabeled nucleic acid precursor [³H]hypoxanthine according to the method described in Desjardins, R. E. et al., 1979, Antimicrob. Agents Chemother., 16: 710-718.

The antimalarial effect of Compound K-I, an exemplary amine borane compound of the present invention, was determined as described above. Compound K-I was dissolved in DMSO and eleven samples of concentrations ranging from 0.5 to 100 μg/ml (final concentrations) were distributed in duplicate in 96-well culture plates and dried in a laminar flow hood. The in vitro response of the parasite was determined by the isotopic microtest developed by Desjardins et al. Infected erythrocytes were suspended in the complete RPMI 1640 medium with 10% fetal bovine serum at a 1.0-2.5% hematocrit media. The suspension (200 μl) was distributed into each well. Parasitemia was adjusted to 0.6% by adding fresh uninfected erythrocytes if the initial parasitemia was ≧1%. Parasite growth was assessed after incubation at 37° C. for 24 hours by adding ³H-hypoxanthine. The cells were incubated for further 15 hours, and then collected by filtration on glass fiber filters and radioactivity was counted. The mean values for parasite control uptake and non-parasitized erythrocyte control uptake of [G-³H]hypoxanthine were calculated from the disintegrations per minute. The IC₅₀ was determined by non-linear regression fitting of the data using the Sigmaplot computer program.

The results obtained for the anti malarial effect of Compound K-I are presented in Table 4 below and in FIG. 2. Table 4 presents the radioactivity, given in counts per minute (cpm), recorded for a parasite sample for each of the tested doses (given in μM/ml). A clear dose-dependent effect is seen. As can be seen in FIG. 2, the measured radioactivity, expressed in counts per minute (cpm), which is indicative of the live parasite, diminishes rapidly as a dose of the amine borane increases, indicating a strong and does-dependent anti malarial effect. T

TABLE 4

| μg/ml | cpm |
|---|---|
| 0 | 3500 |
| 0.3 | 3491 |
| 1 | 3335 |
| 3 | 2836 |
| 9 | 959 |
| 26.8 | 33 |
| 80.6 | 13 |

### Example 4

### Toxicity Studies

Toxicity Studies in Fish:

Compound K-I, an exemplary amine-borane of the present invention, was tested for its toxicity in fish as follows: hybrid Tilapia of the family Cichlidae, order Perciformes (50 grams) vaccinated against Streptococcus, parasites free, were kept (10 fish/100 liter) at a constant temperature of 21° C.

A stock solution of Compound K-I in DMSO (10 mg/liter) was added to the tanks and spread equally in the water so as to achieve the following final concentrations: 3 mg/liter (equivalent to the MIC value of KI against S. parasitica T-1 as determined by the macrodilution method), 10 mg/liter, and 30 mg/liter (10 times of the MIC value). DMSO alone (30 mg/liter) was added to the tanks and served as control.

Fish mortality was recorded 24 and 48 hours after addition of the compound as described in “Toxicity test procedures for fish”, Standard Methods for the Examination of Water and Wastewater, 16t Ed., 1985, American Public Health Association; Washington, D.C., USA, Franson, A H. (Ed.), pp: 810-819.

Water parameters were further measured 24 and 48 hours post addition. The following results were obtained: NH₄-0 mg/liter; NO₃-0.5 mg/liter; pH 8.00; Cl⁻385 mg/liter. These results indicate no change in water quality throughout the experiment.

The results of the toxicity assays of Compound K-I to Tilapia are presented in Table 5 below. The results are presented as percent mortality out of ten fish (10 fish per assay), at the three tested concentrations of the compound.

TABLE 5

| Duration | K-I | K-I | K-I | DMSO |
|---|---|---|---|---|
| of exposure | 3 mg/liter | 10 mg/liter | 30 mg/liter | 30 mg/liter |
| 24 h | 0% | 0% | 10% | 0% |
| 48 h | 0% | 0% | 10% | 0% |

As can be see in Table 5, Compound K-I was found to be non toxic to the fish in concentration of 3 and 10 mg/liter. Minor toxicity was observed in a higher concentration. While the MIC of Compound K-I against the parasite Saprolegnia parasitica, which causes a severe disease in fish, is 1-3 mg per liter (see, Table 1, column 10, entry 9), these results clearly indicate that at these active concentrations and higher the compound is safe for use in fish.

Acute Toxicity Studies in Rats:

The acute toxicity of the novel compounds described herein was determined according to the method described by Falk et al. [27]. Briefly, male ICR mice weighing about 30 grams were injected through the tail vein with various doses of exemplary amine-borane compounds as presented herein and Fungizone (amphotericin B deoxycholate micellar formulation, Bristol-Myers-Squibb, Dublin, Ireland) as control. Each dosage form (Fungizone 0.1 mg/ml; and exemplary amine-borane compounds 1-2 mg/ml in saline) was administered intravenously as single bolus injections (0.12 ml) of the same dose every 10 minutes to a group of three mice until death was observed. The survival of mice that received the maximal tolerated dose (MTD) was monitored for 8 days.

To study the safety of the compounds of the present invention, the MTD that did not kill ICR mice within the 8 days of the experimental period was determined for two exemplary compounds, namely Compound K-I and Compound K-I₂. using Fungizone as a control. The average calculated MTD for both compounds were 121.9 and 73.1 μmol/kg, respectively. The MTD for Fungizone was 2.6 μmol/kg. No changes in clinical signs, body weights, and the gross necropsy (on day 8) of mice that received these dosages were observed. These MTD values were well above that of the in vitro MIC values obtained with Compound K-I (see, Table 1), and the ratio between the MTD and MIC values are comparable to those obtained with amphotericin B, indicating reduced toxicity and therapeutic potential.

Multiple Dose Toxicity (Chronic Toxicity):

The compounds described herein are tested for chronic toxicity. The safety of a 0.2 ml intravenous injection of five consecutive daily therapeutic doses of each compound of the present invention is examined with a group of 10 mice. Survival is monitored for up to 30 days.

### Example 5

### Histopathological Evaluation

The histopathological profile of the compounds described is studied. The tested compounds are administered intravenously by single-bolus injections of 0.2 ml for 5 consecutive days to groups of 10 male albino BALB/c immunocompetent non-infected mice weighing 20-25 grams each. The dose is determined according to the MTD as described hereinabove. Seven day following the first injection mice are sacrificed by CO₂ asphyxiation and the internal organs/tissues are removed, weighed, and handled as follows:

Organs/tissues are fixed in 10% neutral-buffered formalin. Tissues are processed, embedded in paraffin, sectioned at 5-6 μm slices, and stained with hematoxylin and eosin (H&E) for microscopic examination that is carried out by a board certified toxicological pathologist blinded to the identity of the treatment group, and scored for histopathological changes according to the best practices guideline for toxicological histopathology [22].

### Example 6

### Therapeutic Efficacy Study in Animal Models

The efficacy of the compounds described herein is tested in mouse models of systemic candidiasis [21] and invasive aspergillosis [23]. In all the experiments yeasts or spores are injected to the tail vein of male albino Balb/c mice (20±3 grams each) by single bolus of 0.1 ml suspension. The inoculum is about 10⁴ cells per animal from a 24 hours (Candida) or 5-7 days (Aspergillus) culture on Sabouraud Dextrose Agar media incubated at 30° C. Yeasts and spore concentration is determined by hematocytometer count. Viable counts are measured as colony forming units (CFU) on Sabouraud Dextrose Agar media after 2-5 days of incubation at 30° C.

In addition to the mortality, multiplication in the target organs (kidneys in candidiasis and lungs in aspergillosis) as measured by CFU of homogenized organs is monitored. In the murine aspergillosis model a temporal immunosuppression is needed in order to achieve infection, which is afforded by intravenous injection of 200 mg/kg cyclophosphamide (Sigma) 48 hours prior to the infection.

Mice infected as described above are treated with the compounds described herein at various doses. Ten infected mice are used for each treatment. Each group is maintained in a separate cage. Treatment begins 24 hours after the infection by injection of a single bolus (0.2 ml) of each compound for five consecutive days. A control group of 10 infected mice treated only with saline is included for control. The number of surviving animals in each group is recorded daily over a period of 30 days. The results of the survival data are analyzed using the Kolmogorov-Smirnov goodness of fit procedure.

### Example 7

### Cytotoxicology Assay

The cytotoxicity of the compounds described herein is determined by the MTT assay. The MTT Cell Proliferation Assay is based on the cleavage of the yellow tetrazolium salt MTT to purple formazan crystals by metabolic active cells [24-25]. This cellular reduction involves the pyridine nucleotide cofactors NADH and NADPH [26]. The formazan crystals formed are solubilized and the resulting colored solution/is quantified using a scanning multi-well spectrophotometer (ELISA reader). An increase or decrease in cell number results in a concomitant change in the amount of formazan formed, indicating the degree of cytotoxicity caused by the tested compound.

TABLE 1

| No. | Code | Name | Structure | A | B | C | D | E | F | G | H | I |
|---|---|---|---|---|---|---|---|---|---|---|---|---|
| 1 | K-A | 1-dimethylaminomethyl- cyclopent-2-enol cyanoborane | 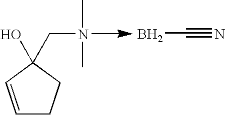 | 1388 | 2776 | 2776 | 500^{c} | >500^{c} | | | | |
| 2 | K-B | (2-hydroxy-2-phenyl- ethyl)-dimethyl-amine cyanoborane | 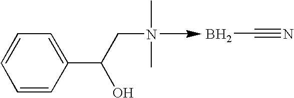 | 245 | >245 | >2450 | >500^{c} | >500^{c} | | | | |
| 3 | K-C | ethyl-dimethyl-amine cyanoborane | 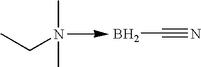 | 4460 | 4460 | 4460 | 500^{c} | 500^{c} | | | | |
| 4 | K-D | but-3-enyl-dimethyl- amine cyanoborane | 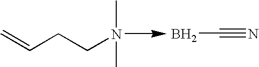 | 470 | 3620 | >3620 | 500^{c} | 500^{c} | | | | >3620 |
| 5 | K-E | trimethyl-amine cyanodibromoborane | 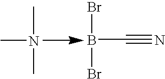 | 1970 | >1970 | >1970 | 500^{c} | >500^{c} | | | | |
| 6 | K-F | trimethyl-amine cyanoborane | 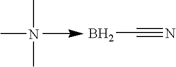 | 5100 | >5100 | >5100 | | | | | | 5100 |
| 7 | K-G | butyl-dimethyl-amine cyanoborane | 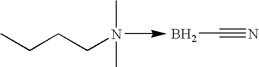 | 3570 | >3570 | >3570 | | | | | | >3570 |
| 8 | K-H | pentyl-dimethyl-amine cyanoborane | 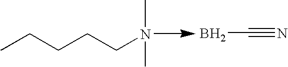 | 810 | 3240 | 3240 | | | | | | 3240 |
| 9 | K-I | dimethyl-undecyl-amine cyanoborane | 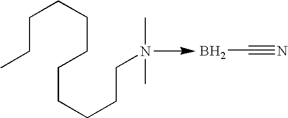 | 32.5 | 32.5 | 32.5 | 7.8^{c} | 15.6^{c} | | | 1-3 | 32.5 |
| 10 | K-I₁ | dimethyl-undecyl-amine cyanobromoborane | 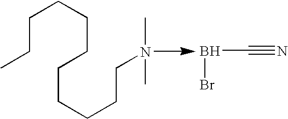 | 49 | 49 | 49 | 7.8^{c} | 7.8^{c} | 7.8^{c} | 15.6 | | 131 |
| 11 | K-I₂ | dimethyl-undecyl-amine cyanodibromoborane | 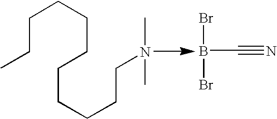 | 39 | 39 | 39 | 15.6^{c} | 15.6^{c} | 32^{c} | 32 | | 79 |
| 12 | K-I₃ | dimethyl-undecyl-amine cyanofluorobromoborane | 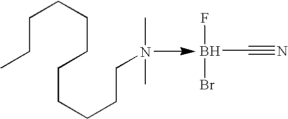 | 7.8 | | | | | | | | |
| 13 | K-J | N,N,N′,N′-tetramethyl- decane-1,10-diamine cyanoborane | 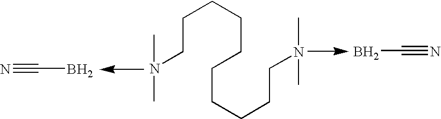 | 408 | 408 | 817 | 32^{c} | 65^{c} | | | | 817 |
| 14 | K-J₁ | N,N,N′,N′-tetramethyl- decane-1,10-diamine bis- cyanobromoborane | 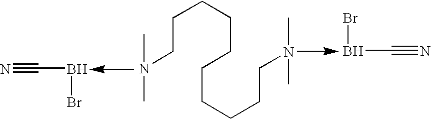 | 140 | 140 | 140 | 65^{c} | 65^{c} | 65^{c} | 65 | | |
| 15 | K-J₂ | N,N,N′,N′-tetramethyl- decane-1,10-diamine bis- cyanodibromoborane | 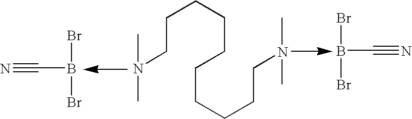 | 104 | 104 | 104 | 65^{c} | 65^{c} | 65^{c} | 65 | | |
| 16 | K-J₃ | N,N,N′,N′-tetramethyl- decane-1,10-diamine bis- carboxyborane | 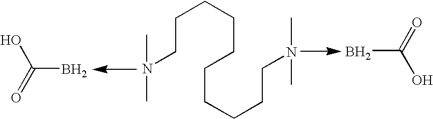 | 188 | 188 | 907 | 65^{c} | 31.2^{c} | 65^{c} | 65 | | |
| 17 | K-K | dimethyl- trimethylsilanylmethyl- amine cyanoborane | 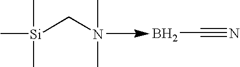 | 2940 | 2940 | 2940 | | | | | | 2940 |
| 18 | K-L | dodecyl-dimethyl-amine cyanoborane | 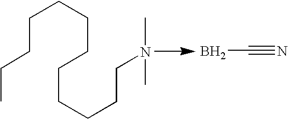 | 61 | 61 | 61 | 65^{c} | 15.6^{c} | 7.8^{c} | 32 | | |
| 19 | K-M | N,N,N′,N′-tetramethyl- dodecane-1,12-diamine bis-cyanoborane | 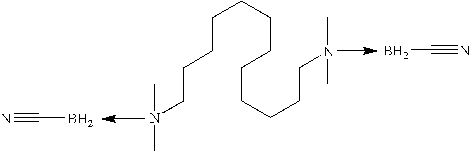 | 96 | 194 | 46 | >250^{c} | >250^{c} | 125^{c} | 250 | | |
| 20 | K-N | N,N,N′,N′-tetramethyl- tetradecane-1,14-diamine bis-cyanoborane | 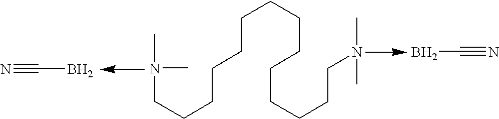 | 43 | 43 | 43 | 7.8^{c} | 15.6^{c} | | | | 86 |
| 21 | K-O | 1-dimethylamino-2- methyl-octan-2-ol cyanoborane | 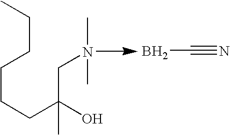 | 2210 | 1100 | 1100 | >250^{c} | >250^{c} | | | | |
| 22 | K-P | dimethyl-nonyl-amine cyanoborane | 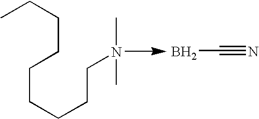 | 150 | 150 | 300 | | | | | | 300 |
| 23 | K-Q | dimethyl-tridecyl-amine cyanoborane | 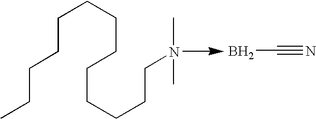 | 58 | 58 | 117 | | | | | | 58 |
| 24 | K-R | dimethyl-pentadecyl- amine cyanoborane | 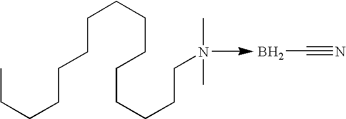 | 53 | 53 | 106 | | | | | | 53 |
| 25 | K-S | heptadecyl-dimethyl- amine cyanoborane | 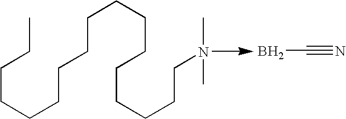 | | | | | | | | | |
| 26 | K-T | 1-dimethylamino- dodecan-2-ol cyanoborane | 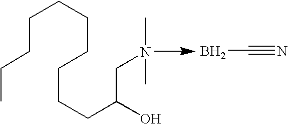 | | | | | | | | | |
| 27 | K-U | 1-dimethylamino- undecan-2-ol cyanoborane | 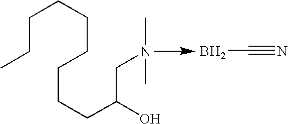 | 492 | 492 | 983 | | | | | | 492 |
| 28 | K-I₄ | dimethyl-undecyl-amine cyanofluoroborane | 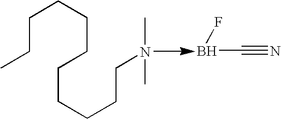 | 64 | 64 | 64 | | | | | | 129 |
| 29 | K-V | hex-5-enyl-dimethyl- amine cyanoborane | 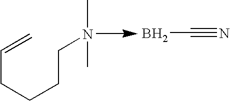 | 94 | 94 | 94 | | | | | | 94 |
| 30 | CAF1 | Amphotericin B | | 0.54 | 0.27 | 0.27 | | | | | | 0.5 |
| 31 | CAF2 | Fluconazole | | 1.6 | 1.6 | 1.6 | | | | | | >208 |

^{c}Values given in μg per liter.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application, shall not be construed as an admission that such reference is available as prior art to the present invention.

### REFERENCES CITED BY NUMERALS

### (Other References are Cited in the Text)

[1] Hall, I. H.; Starnes, C. O.; Mcphail, A. T.; Wisian-Neilson, P.; Das, M. K. Harchelroad, F.; Jr., Spielvogel, B. F. J. Pharm. Sci. 1980, 69(9), 1025.
[2] Kemp, B.; Kalbag, S.; Geanangel, R. A., Inorganic Chemistry (1984), 23(20), 3063-5.
[3] Hall, I. H., Hall, E. S., Chi, L. K., Shaw, B. R., Sood, A. & Spielvogel, B. F. (1992). Antineoplastic activity of boron-containing thymidine nucleosides in Tmolt3 leukemic cells. Anticancer Res 12, 1091-1097.
[4] Miller III, M. C.; A. Sood; Spielvogel, B. F.; Hall, I. H. Appl. Organometal. Chem. 1998, 12, 87.
[5] Hall, I. H.; Starnes, C. O.; Spielvogel, B. F.; Wisian-Neilson, P.; Das, M. K.; Wojnowich, L. J. Pharm. Sci. 1979, 68(6), 685.
[6] Spielvogel, B. F.; Wojnowich, L.; Das, M. K.; McPhail, A. T.; Hargrave, K. D.; J. Am. Chem. Soc. 1976, 98(18), 5702.
[7] Miller III, M. C.; Sood, A., Spielvogel, B. F.; Hall, I. H. Arch. Pharm. Med. Chem. 1998, 331, 153.
[8] Miller III, M. C.; Woods, C. M.; Murphy, M. E.; Elkins, A.; Spielvogel, B. F.; Hall, I. H. Biomed. & Pharmacother 1998, 52, 169.
[9] Hall, I. H., Chen, S. Y., Rajendran, K. G., Sood, A., Spielvogel, B. F. & Shih, J. (1994). Hypolipidemic, anti-obesity, anti-inflammatory, anti-osteoporotic, and anti-neoplastic properties of amine carboxyboranes. Environ Health Perspect 102 Suppl 7, 21-30.
[10] Spielvogel, B. F.; Das, M. K.; McPhail, A. T.; Onan, K. D.; Hall, I. H., J. Am. Chem. Soc. 1980, 102, 6344.
[11] Murphy, M. E.; Elkins, A. L.; Shrewsbury, R. P.; Sood, A.; Speilvogel, B. F. Hall, I. H. Metal Based Drugs 1996, 3, 31.
[12] Hall, I. H.; Burnham, B. S.; Chen, S. Y.; Sood, A.; Spielvogel, B. F., Morse, K. W. Metal Based Drugs 1995, 2(1), 1.
[13] Hall, I. H.; Williams, W. L.; Jr.; Gilbert, C. J.; McPhail, A. T.; Spielvogel, B. F. J. Pharm. Sci. 1980, 73(7), 973.
[14] Hall, I. H.; Gilbert, C. J.; McPhail, A. T.; Morse, K. W.; Hassett, K.; Speilvogel, B. F. J. Pharm. Sci. 1985, 74(7), 755.
[15] Sood, C. K.; Sood, A.; Speilvogel, B. F.; Yousef, J. A.; Burnham, B.; Hall, I. H. J. Pharm. Sci. 1991, 80(12), 1133.
[16] Dembitsky, V. M.; Srebnik, M., Tetrahedron 2003, 59, 579.
[17] Takrouri et al., Organometallics (2004), 23(11), 2817-2820.
[18] National Committee for Clinical Laboratory Standards (1997). National Committee for Clinical Laboratory Standards Reference method for broth dilution antifungal susceptibility testing of yeasts. (M27-A, A. s., Ed), Wayne, Pa.
[19] National Committee for Clinical Laboratory Standards (2002). Reference method for broth dilution antifungal susceptibility testing of filamentous fungi. In: Approved Standard—Second Edition M38-A). National Committee for Clinical Laboratory Standards, Wayne, Pa.
[20] Ephros M, Waldman E, Zilberstein D. 1997. Pentostam induces resistance to antimony and the preservative chlorocresol in L. donovani promastigotes and axenically grown amastigotes. Antimicrob Agents Chemother. 41: 1064-68.
[21] Falk, R., Domb, A. J. & Polacheck, I. (1999). A novel injectable water-soluble amphotericin B-arabinogalactan conjugate. Antimicrob Agents Chemother 43, 1975-1981.
[22] Crissman, J. W., Goodman, D. G., Hildebrandt, P. K., Maronpot, R. R., Prater, D. A., Riley, J. H., et al. (2004). Best practices guideline: toxicologic histopathology. Toxicol Pathol 32, 126-131.
[23] Falk, R., Grunwald, J., Hoffman, A., Domb, A. J. & Polacheck, I. (2004). Distribution of amphotericin B-arabinogalactan conjugate in mouse tissue and its therapeutic efficacy against murine aspergillosis. Antimicrob Agents Chemother 48, 3606-3609.
[24] Vistica, D. T. et al. (1991) Cancer Res. 51, 2515-2520.
[25] Maehara, Y. et al. (1986) Eur. J. Cancer. Clin. Oncol. 23, 273-276
[26] Beridge, M. V. & Tan, A. S. (1993) Arch. Biochem. Biophys. 303, 474
[27] Falk, R.; Domb, A. J.; Polacheck, I. A novel injectable water-soluble amphotericin B-arabinogalactan conjugate. Antimicrob. Agents Chemother. 1999, 43, 1975-1981.

1-49. (canceled)

50. A method of treating a medical condition associated with a eukaryotic pathogenic microorganism, the method comprising administering to a subject in need thereof a therapeutically effective amount of an amine-borane compound.

51. The method of claim 50, wherein said pathogenic microorganism is a drug-resistant microorganism.

52. The method of claim 50, wherein said medical condition is selected from the group consisting of a fungal infection, a protozoan infection, malaria and leishmaniasis.

53. A pharmaceutical composition comprising, as an active ingredient, an amine-borane compound and a pharmaceutically acceptable carrier, the pharmaceutical composition being packaged in a packaging material and identified in print, in or on said packaging material, for use in the treatment of a medical condition associated with a eukaryotic pathogenic microorganism.

54. The pharmaceutical composition of claim 53, wherein said pathogenic microorganism is a drug-resistant microorganism.

55. A method of treating a medical condition associated with a eukaryotic pathogenic, drug-resistant microorganism, the method comprising administering to a subject in need thereof a therapeutically effective amount of an amine-borane compound.

56. A method of reducing the load of a eukaryotic microorganism in a substrate, the method comprising applying to the substrate an antimicrobial effective amount of an amine-borane compound.

57. The method, use or composition of claim 56, wherein said microorganism is a resistant microorganism.

58. An article-of-manufacturing comprising a product and an antimicrobial effective amount of an amine-borane compound, said amine-borane compound being for reducing a load of an eukaryotic microorganism in said product.

59. A method of treating a medical condition associated with a pathogenic microorganism, the method comprising administering to a subject in need thereof a therapeutically effective amount of an amine-borane compound having the general Formula I or II: 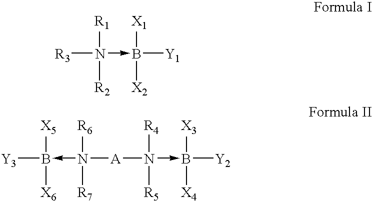
or a pharmaceutically acceptable salt thereof,
wherein:
Y₁, Y₂ and Y₃ are each independently selected from the group consisting of a cyano group (—C≡N), a —C(═O)Ra group, amine and alkyl, whereas Ra is selected from the group consisting of hydrogen, halo, hydroxy, alkoxy, thiohydroxy, thioalkoxy, aryloxy, thioaryloxy, thiol and amine;
X₁-X₆ are each independently selected from the group consisting of hydrogen, alkyl, halo, cycloalkyl, and aryl;
R₁-R₇ are each independently selected from the group consisting of hydrogen, alkyl, alkenyl, alkynyl, cycloalkyl and aryl or, alternatively, two of R₁-R₃, R₄ and R₅ and/or R₆ and R₇ form a carbocyclic ring; and
A is a substituted or non-substituted, saturated or non-saturated hydrocarbon having from 1 to 20 carbon atoms.

60. A pharmaceutical composition comprising, as an active ingredient, an amine-borane compound having the general Formula I or II: 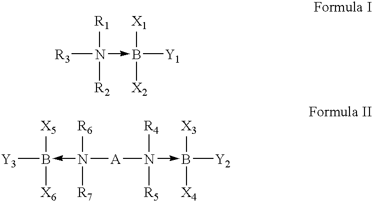
or a pharmaceutically acceptable salt thereof,
wherein:
Y₁, Y₂ and Y₃ are each independently selected from the group consisting of a cyano group (—C≡N), a —C(═O)Ra group, amine and alkyl, whereas Ra is selected from the group consisting of hydrogen, halo, hydroxy, alkoxy, thiohydroxy, thioalkoxy, aryloxy, thioaryloxy, thiol and amine;
X₁-X₆ are each independently selected from the group consisting of hydrogen, alkyl, halo, cycloalkyl, and aryl;
R₁-R₇ are each independently selected from the group consisting of hydrogen, alkyl, alkenyl, alkynyl, cycloalkyl and aryl or, alternatively, two of R₁-R₃, R₄ and R₅ and/or R₆ and R₇ form a carbocyclic ring; and
A is a substituted or non-substituted, saturated or non-saturated hydrocarbon having from 1 to 20 carbon atoms;
and a pharmaceutically acceptable carrier, the composition being packaged in a packaging material and identified in print, in or on said packaging material, for use in the treatment of said medical condition.

61. The method of claim 59, wherein said pathogenic microorganism is a drug-resistant microorganism.

62. The method of claim 59, wherein said medical condition is selected from the group consisting of a bacterial infection, a fungal infection, a protozoan infection, malaria and leishmaniasis.

63. The method of claim 59, wherein Y₁ is selected from the group consisting of a cyano group (—C≡N) and a —C(═O)Ra group.

64. The method of claim 63, wherein at least one of X₁ and X₂ is halo.

65. The method of claim 59, wherein each of R₁-R₃ is alkyl.

66. The method of claim 59, wherein each of Y₂ and Y₃ is a cyano group (—C≡N).

67. The method of claim 59, wherein each of Y₂ and Y₃ is a —C(═O)Ra group.

68. The method of claim 59, wherein at least one of X₃-X₆ is halo.

69. A method of treating a medical condition associated with a pathogenic, drug-resistant microorganism, the method comprising administering to a subject in need thereof a therapeutically effective amount of an amine-borane compound having the general Formula I or II: 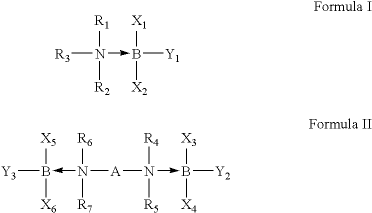
or a pharmaceutically acceptable salt thereof,
wherein:
Y₁, Y₂ and Y₃ are each independently selected from the group consisting of a cyano group (—C≡N), a —C(═O)Ra group, amine and alkyl, whereas Ra is selected from the group consisting of hydrogen, halo, hydroxy, alkoxy, thiohydroxy, thioalkoxy, aryloxy, thioaryloxy, thiol and amine;
X₁-X₆ are each independently selected from the group consisting of hydrogen, alkyl, halo, cycloalkyl, and aryl;
R₁-R₇ are each independently selected from the group consisting of hydrogen, alkyl, alkenyl, alkynyl, cycloalkyl and aryl or, alternatively, two of R₁-R₃, R₄ and R₅ and/or R₆ and R₇ form a carbocyclic ring; and
A is a substituted or non-substituted, saturated or non-saturated hydrocarbon having from 1 to 20 carbon atoms.

70. A method of reducing the load of a microorganism in a substrate, the method comprising applying to the substrate an antimicrobial effective amount of an amine-borane compound having the general Formula I or II: 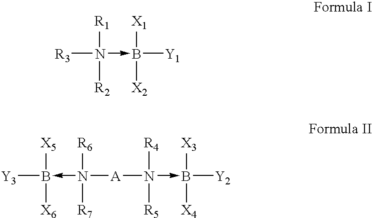
or a pharmaceutically acceptable salt thereof,
wherein:
Y₁, Y₂ and Y₃ are each independently selected from the group consisting of a cyano group (—C≡N), a —C(═O)Ra group, amine and alkyl, whereas Ra is selected from the group consisting of hydrogen, halo, hydroxy, alkoxy, thiohydroxy, thioalkoxy, aryloxy, thioaryloxy, thiol and amine;
X₁-X₆ are each independently selected from the group consisting of hydrogen, alkyl, halo, cycloalkyl, and aryl;
R₁-R₇ are each independently selected from the group consisting of hydrogen, alkyl, alkenyl, alkynyl, cycloalkyl and aryl or, alternatively, two of R₁-R₃, R₄ and R₅ and/or R₆ and R₇ form a carbocyclic ring; and
A is a substituted or non-substituted, saturated or non-saturated hydrocarbon having from 1 to 20 carbon atoms.

71. The method of claim 70, wherein said microorganism is a resistant microorganism.

72. An article-of-manufacturing comprising a product and an antimicrobial effective amount of an amine-borane compound having the general Formula I or II: 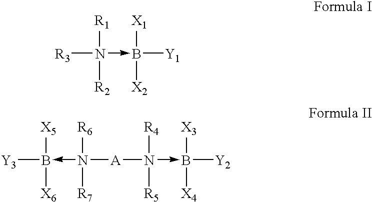
or a pharmaceutically acceptable salt thereof,
wherein:
Y₁, Y₂ and Y₃ are each independently selected from the group consisting of a cyano group (—C≡N), a —C(═O)Ra group, amine and alkyl, whereas Ra is selected from the group consisting of hydrogen, halo, hydroxy, alkoxy, thiohydroxy, thioalkoxy, aryloxy, thioaryloxy, thiol and amine;
X₁-X₆ are each independently selected from the group consisting of hydrogen, alkyl, halo, cycloalkyl, and aryl;
R₁-R₇ are each independently selected from the group consisting of hydrogen, alkyl, alkenyl, alkynyl, cycloalkyl and aryl or, alternatively, two of R₁-R₃, R₄ and R₅ and/or R₆ and R₇ form a carbocyclic ring; and
A is a substituted or non-substituted, saturated or non-saturated hydrocarbon having from 1 to 20 carbon atoms.
